(19) United States
(12) Patent Application Publication
(10) Pub. No.: US 2019/0125912 A1
(43) Pub. Date: May 2, 2019

(54) REMOVABLE CARTRIDGES FOR USE WITH PROCESS MONITORING SYSTEMS, AND SYSTEMS COMPRISING SAME

(71) Applicant: 3M INNOVATIVE PROPERTIES COMPANY, St. Paul, MN (US)

(72) Inventors: G. MARCO BOMMARITO, STILLWATER, MN (US); BARRY W. ROBOLE, WOODVILLE, WI (US)

(21) Appl. No.: 16/094,545

(22) PCT Filed: Apr. 14, 2017
(86) PCT No.: PCT/US2017/027587

§ 371 Date: Oct. 18, 2018

Related U.S. Application Data
(60) Provisional application No. 62/326,329, filed on Apr. 22, 2016.

(51) Int. Cl. A61L 2/28; A61B 1/12; A61L 2/18; G01N 31/22

(52) U.S. Cl. CPC A61L 2/28; A61B 1/123; A61B 2090/702; G01N 31/221; A61L 2/18

(57) ABSTRACT

A cartridge for use with a process monitoring system. The process monitoring system can include an adapter comprising a first fluid pathway and configured to be positioned in fluid communication with a reprocessing system. At least a portion of the cartridge can be configured to be removably received in a receptacle of the adapter. The cartridge can include a second fluid pathway configured to be positioned in fluid communication with the first fluid pathway of the adapter when at least a portion of the cartridge is positioned in the receptacle of the adapter. The cartridge can further include at least one indicator positioned on the cartridge in fluid communication with the second fluid pathway of the cartridge.

### FIELD

The present disclosure generally relates to removable cartridges for use with process monitoring systems, and particularly, for use with process monitoring systems configured to monitor endoscope reprocessing systems.

### BACKGROUND

Endoscopy procedures play a beneficial role in the prevention, diagnosis and treatment of disease. Endoscopy procedures are performed using complex, reusable, flexible instruments that, when inserted into the body, may become heavily contaminated with patient biomaterial and microorganisms, including potential pathogens. Careful reprocessing of flexible endoscopes between patients is critical to reducing the risk of cross-contamination and the possible transmission of pathogens.

Flexible endoscopes are rated as semi-critical according to the Spaulding classification for medical devices and therefore it is required that these devices be decontaminated by high-level disinfection. Thus, it is recommended that both endoscopes and reusable accessories be frequently visually inspected in the course of their use and reprocessing, including before, during and after use, as well as after cleaning and before high-level disinfection. However, a visually based method of verification has severe limitations when applied to flexible endoscopes because the complex, narrow lumens in these devices cannot be directly visually inspected.

Automated endoscope reprocessors (AERs) are used to clean and disinfect flexible endoscopes to a level that mitigates transmission of pathogenic organisms and disease between patients who are subject to an endoscopic procedure. Typically, the only information available to a user is the parametric information provided by the AER equipment itself which consists primarily of time and temperature information. The AER does not monitor chemical parameters capable of establishing the effectiveness of the disinfection cycle.

### SUMMARY

Some aspects of the present disclosure provide a cartridge for use with a process monitoring system. The process monitoring system can include an adapter comprising a first fluid pathway and configured to be positioned in fluid communication with a reprocessing system. At least a portion of the cartridge can be configured to be removably received in a receptacle of the adapter. The cartridge can include a second fluid pathway having an inlet and an outlet configured to be positioned in fluid communication with the first fluid pathway of the adapter when at least a portion of the cartridge is positioned in the receptacle of the adapter, such that the second fluid pathway of the cartridge is in fluid communication with the first fluid pathway of the adapter and fluid flow through the first fluid pathway of the adapter is at least partially diverted through the second fluid pathway of the cartridge when at least a portion of the cartridge is received in the receptacle of the adapter. The cartridge can further include at least one indicator positioned on the cartridge in fluid communication with the second fluid pathway of the cartridge. Other features and aspects of the present disclosure will become apparent by consideration of the detailed description and accompanying drawings.

### BRIEF DESCRIPTION OF THE DRAWINGS

FIG. 1 is a schematic isometric exploded view of a process monitoring system according to one embodiment of the present disclosure.

FIG. 2 is a schematic top assembled view of the process monitoring system of FIG. 1.

FIG. 3 is an endoscope reprocessing system according to one embodiment of the present disclosure.

FIG. 4 is an endoscope reprocessing system according to another embodiment of the present disclosure.

FIGS. 5-15 are schematic diagrams of process monitoring systems according tovarious embodiments of the present disclosure.

FIG. 16 is an exploded isometric view of a process monitoring system according to another embodiment of the present disclosure.

FIG. 17 is an assembled isometric view of the process monitoring system of FIG. 16.

FIG. 18 is a cross-sectional view of the process monitoring system of FIGS. 16-17,taken along line 18-18 of FIG. 17.

FIG. 19 is a top plan view of a cartridge according to another embodiment of the present disclosure.

FIG. 20 is a top plan view of a cartridge according to another embodiment of the present disclosure.

FIG. 21 is an exploded isometric view of a process monitoring system according to another embodiment of the present disclosure.

FIG. 22 is an assembled isometric view of the process monitoring system of FIG. 21.

FIG. 23 is a cross-sectional view of the process monitoring system of FIGS. 21-22,taken along line 23-23 of FIG. 22.

FIG. 24 is a cross-sectional view of the cartridge of the process monitoring system of FIGS. 21-22, taken along line 24-24 of FIG. 22.

FIG. 25 is a cross-sectional view of a cartridge according to another embodiment of the present disclosure.

### DETAILED DESCRIPTION

The present disclosure generally relates to cartridges and adapters for use with process monitoring systems, and process monitoring systems comprising such cartridges and adapters.

Definitions

The term “a”, “an”, and “the” are used interchangeably with “at least one” to mean one or more of the elements being described.

The term “and/or” means either or both. For example “A and/or B” means only A, only B, or both A and B.

The terms “including,” “comprising,” or “having,” and variations thereof, are meant to encompass the items listed thereafter and equivalents thereof as well as additional items.

Unless specified or limited otherwise, the terms “connected” and “coupled” and variations thereof are used broadly and encompass both direct and indirect connections and couplings.

Process monitoring systems of the present disclosure, and elements thereof, can be used to monitor the effectiveness of a variety of reprocessing systems, including various cleaning, disinfecting, and/or sterilization processes or systems. For example, in some embodiments, process monitoring systems of the present disclosure can be used to monitor an endoscope reprocessing system. Such an endoscope reprocessing system can include, but is not limited to, an automated endoscope reprocessor (AER), an endoscope cleaning reprocessor (ECR), a liquid chemical sterilization (LCS) system, or the like, or a combination thereof. By way of example only, the process monitoring systems of the present disclosure can be particularly useful for monitoring the effectiveness of a disinfection cycle provided by an AER. As a result, the cartridges, adapters, and systems of the present disclosure are sometimes described herein with reference to use with an AER. However, it should be understood that the cartridges, adapters, and systems of the present disclosure can be used in monitoring other endoscope reprocessing systems, as well as other cleaning, disinfecting, and/or sterilization processes or systems.

Cartridges of the present disclosure can be a consumable component of the system and can be configured to be removably received in a receptacle of an adapter. Adapters of the present disclosure can provide a means for effectively connecting (i.e., for fluid communication) the cartridge to a reprocessing system that is to be monitored for its effectiveness. In some embodiments, the adapter can be referred to as a manifold.

Cartridges of the present disclosure can include various features of the process challenge devices described WO2016/164329, filed Apr. 5, 2016 (Attorney Docket No. 76311WO003), which is incorporated herein by reference in its entirety.

In some embodiments, the adapter can include a first fluid pathway that is configured to be positioned in fluid communication with a reprocessing system. Cartridges can be configured such that at least a portion thereof can be removably received in a receptacle of the adapter (e.g., in a receptacle of the adapter housing). Such a cartridge can include a second fluid pathway that is configured to be positioned in fluid communication with the first fluid pathway when at least a portion of the cartridge is received in the receptacle of the adapter, e.g., such that at least a portion of the fluid flow through the first fluid pathway of the adapter can be routed or diverted through the cartridge. In such embodiments, when the cartridge is not present (i.e., not received in the receptacle of the adapter), fluid can flow entirely through the first fluid pathway in the adapter. In some cases, it can be advantageous if the fluid flow through the adapter (either alone when no cartridge is present, or when a portion of the fluid is diverted through the cartridge) does not significantly affect the overall resistance of the particular reprocessing system that is to be monitored.

Cartridges of the present disclosure can also include one or more indicators positioned on the cartridge in fluid communication with the second fluid pathway, such that the fluid flow through the cartridge comes into contact with the one or more indicators. Such indicators can include a chemical indicator, a biological indicator, or a combination thereof.

Chemical indicators can be read immediately at the end of reprocessing cycle. The results can indicate that a particular condition was present during the re-processing, such as the presence of a particular chemical or a temperature, and potentially, that the condition was reached for a certain period of time.

Suitable chemical indicators for use with the cartridges and process monitoring systems of the present disclosure can include a colorimetric system to verify a condition of interest (e.g., a minimum effective concentration (MEC), e.g., of a disinfectant liquid) was met. One possible system would be based on the reaction of a commonly used high level disinfectant, ortho-phthalaldehyde with sodium sulfite disposed on a substrate. The reaction forms a sulfite addition product and an equivalent amount of base according to the following reaction:

C₆H₄(CHO)₂+2Na₂SO₃₊2H₂O=>C₆H₄(CH(SO₃Na)OH)₂+2NaOH

If sufficient ortho-phthalaldehyde is present, the increase in pH causes a color change in the pH indicator also disposed on the substrate. When the concentration of ortho-phthalaldehyde is sufficient, the local pH typically rises above 11 and a color change to a deep purple occurs. There are several suitable pH dyes that can be used in this indication. A similar reaction scheme can be used to test MEC for glutaraldehyde (GA) disinfectants, another common class of High Level Disinfection (HLD) chemicals used, e.g., in reprocessing flexible endoscopes. The chemical indication could be also configured to be an integrator, meaning that it will measure not just whether the disinfectant is above a certain concentration but for how long it was at that concentration. This could be done by providing an indicator system where the colorimetric response was proportional to a dosage or contact time. For example, by disposing the indicator system along a wicking strip rather than in a dot, and allowing for capillary action in the wicking material to dictate the flow of disinfectant along the strip, visualization of the colorimetric front along the strip would then become an indication of time as well as the condition of interest. The porosity of the strip would be chosen to achieve to desired movement of disinfectant along the strip for a given cycle duration. The wicking strip could be made of an appropriate membrane or filtration material but it could also be engineered as an additional microfluidic component that forms a monolithic structure along with a challenge channel (described in greater detail below) of the process monitoring system.

Biological indicators, on the other hand, can include a source of biological activity. As such, the response of sources of biological activity to all conditions actually present can be a more direct and reliable test for the effectiveness of a reprocessing method. The source(s) of biological activity employed can be coated directly onto a surface in an indicator chamber of a cartridge of the present disclosure, and/or can be coated onto a substrate that is positioned in the indicator chamber.

After the indicator is exposed to the reprocessing conditions, the sources of biological activity (e.g., spores) can be incubated in a nutrient medium (e.g., that is located in a closed on-board chamber that can be fractured after the reprocessing step) to determine whether any of the sources survived the sterilization process, with source metabolism and/or growth indicating that the reprocessing conditions were insufficient to destroy all of the sources of biological activity.

Nutrient medium used to nourish the sources of biological activity (e.g., spores) following a sterilization procedure can be present throughout the reprocessing procedure but may not be accessible by the sources of biological activity until desired. For example, a frangible pouch, chamber or container can house the medium ‘on board’ separately from the sources of biological activity, and the container can be fractured to put the sources of biological activity and medium in fluid communication with one another, when desired (e.g., after a reprocessing procedure). Nutrients and nutrient media to facilitate the growth of microorganisms are known in the art and can be found, for example, in the “Handbook of Microbiological Media” by Ronald Atlas, published by CRC Press, Boca Raton, Fla. Matner et al. (U.S. Pat. No. 5,073,488), which is incorporated herein by reference in its entirety.

Generally, sources of biological activity (e.g., microorganisms) are chosen to be used in a biological indicator that are resistant to a particular reprocessing procedure. The biological indicators of the present disclosure include a viable quantity, or culture, of one or more known sources of biological activity (e.g., species of microorganism). Such sources of biological activity can be in the form of spores. The test source in the biological indicator is either killed by a successful reprocessing cycle, or survives if the cycle is not adequate for some reason. Spores, rather than the vegetative form of the organisms, are sometimes used at least partly because vegetative microorganisms are known to be relatively easily killed by various processes. Spores can also have superior storage characteristics and can remain in their dormant state for years. As a result, in some embodiments, disinfection of an inoculum of a standardized spore strain can provide a high degree of confidence that inactivation of all microorganisms in a reprocessing cycle has occurred.

By way of example only, the present disclosure describes the one or more sources of biological activity used in the biological sterilization indicator as being “spores;” however, it should be understood that the type of source (e.g., spore) used in a particular embodiment of the biological sterilization indicator is selected for being highly resistant to the particular reprocessing procedure contemplated. Accordingly, different embodiments of the present disclosure may use different sources of biological activity, depending on the process for which the particular embodiment is intended. The term “spores” is used for simplicity, but it should be understood that other sources of biological activity, such as microorganisms (e.g., bacteria, fungi, viruses, etc.), spores (e.g., bacterial, fungal, etc.), enzymes, substrates for enzymatic activity, ATP, microbial metabolites, polynucleotides (e.g., ribozymes, DNA, RNA, fragments thereof, etc.), or a combination thereof, can be used in the biological indicator of the present disclosure.

The phrase “biological activity” generally refers to any specific catalytic process or groups of processes associated with a biological cell. Nonlimiting examples of biological activities include catabolic enzyme activities (e.g., carbohydrate fermentation pathways), anabolic enzyme activities (e.g., nucleic acid, amino acid, or protein synthesis), coupled reactions (e.g., a metabolic pathway), biomolecule-mediated redox reactions (e.g., electron transport systems), and bioluminescent reactions. “Predetermined” biological activity means that the method is directed toward the detection of a specific biological process (e.g., an enzyme reaction) or group of biological processes (e.g., a biochemical pathway). It will be appreciated by a person having ordinary skill in the art that certain predetermined biological activities may be associated with a particular type of cell (e.g., cancer cell or microorganism) or a pathological process.

It may be possible to use spores or weakened/injured spores as the biological indicator. As mentioned above, one of the advantages of using spores in this application is that they are “shelf stable” for long times at room temperature. Germination and growth of the spores is not easily triggered except by design. In some embodiments, it may be possible to simply measure the amount of viable spores present after a reprocessing (e.g., disinfection) cycle and compare it to the predetermined amount of spores placed in a chamber of the cartridge. That difference in the spore population pre and post reprocessing could then be compared to an expected difference for an effective cycle, and within a certain tolerance window, a determination could be made on whether the reprocessing cycle was effective or not (i.e., pass or fail). The measured difference would also quantify the log reduction achieved during the cycle.

If bacterial spores were found to be too resistant to be affected by the reprocessing cycle (e.g., by the disinfectant used in AERs), another potential biological entity useful in this indication could be an appropriate yeast. For example, Saccharomyces cerevisiae is a species of yeast that could be employed in this concept. It is a yeast cell instrumental to winemaking, baking, and brewing and it is one of the most intensively studied eukaryotic model organisms in molecular and cell biology. Rapid detection of the biological indication could be achieved using a florescence based enzymatic reaction. Glucosidase assays using fluorogenic substrates are one such class. For example, β-Glucosidase catalyzes the breakdown of the β-glucosidic linkage in the fluorogenic substrate, β-MUG, to release its component moieties glucose and the fluorescent compound 4-MU. The activity of this enzyme can then be measured as an increase in fluorescence over time from germinated spore suspensions. The reaction is potentially quantitative and could be used to determine the difference from a predetermined initial spore population prior to the initiation of a reprocessing cycle to a final spore population upon completion of the cycle.

Another means of determining the efficacy of a reprocessing cycle may be to measure the kinetics of the increasing fluorescence signal from the viable spores remaining after reprocessing. The pass/fail determination may then be based on how quickly the fluorescent intensity reached a given level. It may also be possible to use colorimetric assays instead of fluorescence based assays, although these may be less sensitive. It may also be possible for the enzymatic assay to drive an electrochemical response. In this mode, rather than integrating light signals, one would either measure changes in potential (coulometric) or current flow (amperometric).

In some embodiments of the present disclosure, the process monitoring system can provide a resistance to volumetric flow (i.e., liquid flow) that mimics the challenge posed by an endoscope (e.g., including a long, narrow lumen). In some embodiments, the resistance to flow can be provided by a challenge channel, which is described in greater detail below. In some embodiments, such a challenge channel can be located in one or both of the removable cartridge and the adapter.

Some embodiments of the present disclosure are directed to an endoscope reprocessing system that includes a process monitoring system of the present disclosure, and a fluid pathway configured to be positioned in fluid communication with the process monitoring system and an endoscope. In such systems, the process monitoring system and the endoscope can be fluidly coupled in series or in parallel. Furthermore, such an endoscope reprocessing system can include a basin for receiving an endoscope. In such embodiments, the adapter (or the process monitoring system as a whole) can be located outside of the basin.

### Illustrated Embodiments

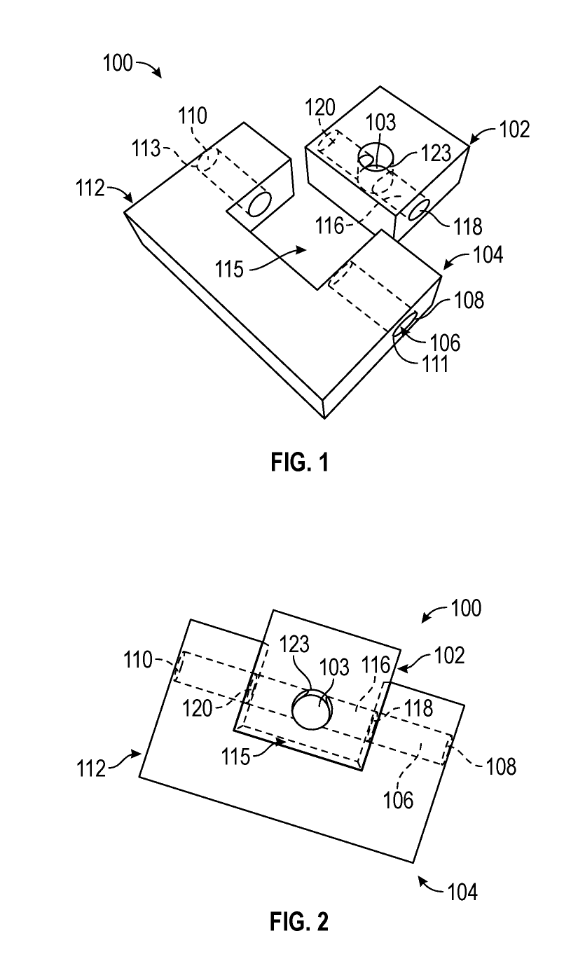

FIGS. 1 and 2 illustrate a process monitoring system 100 according to one embodiment of the present disclosure, including a cartridge (or “test cartridge”) 102 according to one embodiment of the present disclosure, and an adapter 104 according to one embodiment of the present disclosure. The cartridge 102 can include one or more indicators 103 configured to indicate a monitoring result (e.g., a pass or fail) to a user regarding whether a particular reprocessing system or process of interest met the necessary conditions.

As shown in FIGS. 1 and 2, the adapter 104 includes a first fluid pathway 106 that is open-ended (i.e., when not coupled to a reprocessing system), including an inlet 108 and an outlet 110. The first fluid pathway 106 can be configured to be positioned in fluid communication with a reprocessing system via the inlet 108 and the outlet 110. For example, in some embodiments in which the process monitoring system 100 is used with an endoscope reprocessing system (e.g., an automated endoscope reprocessor (AER)), the inlet 108 can be fluidly coupled to a pump, or fluid flow source, e.g., to an AER fluid output connector. In addition, in such embodiments, the outlet 110 can be fluidly coupled to an endoscope, e.g., via an appropriate connection harness. A variety of commercially available connection harnesses can be used, depending on the model and type of endoscope being reprocessed and the endoscope reprocessing system being used, such as those available under the trade designation MEDIVATORS® DSD and SSD Series from Medivators Inc., Minneapolis, Minn.

Such an arrangement as that described above essentially places the process monitoring system 100 (i.e., the adapter 104) in the main fluid pathway of an AER, e.g., downstream of a pump and upstream of an endoscope to be reprocessed. However, this need not be the case, and as described greater detail below, the process monitoring system 100 can be positioned in series with the main fluid pathway at any point with respect to the endoscope (e.g., upstream or downstream); or can be positioned in parallel with the main fluid pathway.

The adapter 104 can further include a housing 112 that can include and define the first fluid pathway 106. For example, as shown in FIG. 1, the housing 112 can include a first opening 111 that defines the inlet 108 of the first fluid pathway 106, and a second opening 113 that defines the outlet 110 of the first fluid pathway 106. As shown in FIGS. 1 and 2, the housing 112 can further include a receptacle 115 dimensioned to receive at least a portion of the cartridge 102. In some embodiments, at least a portion of the cartridge 102 can be received in the receptacle 115 with an audible and/or tactile feedback to the user that informs the user that the cartridge 102 has been fully and properly positioned in the receptacle 115.

By way of example only, in the embodiment of FIGS. 1 and 2, the receptacle 115 is open, and the cartridge 102 is shaped and sized to be slid into the receptacle 115. However, it should be understood that other receptacle and card coupling configurations can be employed—for example, in some embodiments, the housing 112 can include one or more portions that can be movable with respect to one another (e.g., in a clamshell configuration) between an open and closed positioned in order to access the receptacle 115 and to position the cartridge 102 in the receptacle 115.

The cartridge 102 can include a second fluid pathway 116 that is open-ended (i.e., when the cartridge 102 is not coupled to the adapter 104), includes an inlet 118 and an outlet 120, and is configured to be positioned in fluid communication with the first fluid pathway 106 of the adapter 104 when at least a portion of the cartridge 102 is received in the receptacle 115 of the adapter 104. As a result, the second fluid pathway 116 of the cartridge 102 can be in fluid communication with (e.g., can form a portion of) the first fluid pathway 106 of the adapter 104 when the cartridge 102 is at least partially received in the receptacle 115 of the adapter 104, and fluid flow through the first fluid pathway 106 of the adapter 104 can be at least partially diverted through, or moved through, the second fluid pathway 116 of the cartridge 102. In addition, at least a portion of fluid flow in the reprocessing system into which the process monitoring system 100 is incorporated (e.g., from a pump to an endoscope) can be routed through the cartridge 102 before entering the endoscope. The portion of fluid flow that is sampled by the cartridge 102 can vary.

In some embodiments, it can be advantageous to divert or sample only a small portion of the overall fluid flow in order to minimize the overall effect (e.g., added resistance) that the process monitoring system 100 has on the reprocessing system it is monitoring.

In addition, in some embodiments, as shown in FIGS. 1 and 2, when at least a portion of the cartridge 102 is received in the receptacle 115 of the adapter 104, the second fluid pathway 116 of the cartridge 102 can be fluidly coupled to the first fluid pathway 106 such that the inlet 118 and the outlet 120 of the second fluid pathway 116 of the cartridge 102 are located between the inlet 108 and the outlet 110 of the first fluid pathway 106 of the adapter 104, and particularly, between the first and second openings 111 and 113 in the housing 112.

The cartridge 102 can be movable with respect to the adapter 104 between (i) a first position (see FIG. 1) in which the cartridge 102 is not received in the receptacle 115 of the adapter 104, and the second fluid pathway 116 is not in fluid communication with the first fluid pathway 106 of the adapter 104; and (ii) a second position (see FIG. 2) in which the cartridge 102 is at least partially received in the receptacle 115 of the adapter 104, and the second fluid pathway 116 is in fluid communication with the first fluid pathway 106 of the adapter 104.

While not shown in FIGS. 1 and 2 for simplicity, a person of ordinary skill in the art will appreciate that the first and second fluid pathways 106, 116 can include gaskets, seals and/or valves (e.g., at the inlets 108, 118 and outlets 110, 120) to control fluid connection between the first and second fluid pathways 106, 116 and to prevent leaks.

As mentioned above, the cartridge 102 functions as the test portion of the process monitoring system 100 and includes one or more indicators 103 located on the cartridge 102, which can include at least one of a chemical indicator and a biological indicator, as described above. In some embodiments, one or more of the indicators 103 can include a chemical indicator that is configured to determine whether the liquid disinfectant was present at a suitable concentration, for a suitable period of time, and/or was present at a suitable temperature.

In some embodiments, as shown in FIGS. 1 and 2, each indicator 103 can be located in a chamber 123 that is in fluid communication with the second fluid pathway 116, such that fluid moving through the second fluid pathway 116 will contact the indicator 103. In some embodiments, the cartridge 102 can be transparent or can include a transparent window adjacent the chamber 123, such that the indicator 103 is visible or detectable by a reading apparatus, e.g., after being removed from being coupled to the adapter 104.

The cartridge 102 can form a removable and consumable portion of the process monitoring system 100, and the adapter 104 can form a more permanent portion of the process monitoring system 100. In some embodiments, the adapter 104 can be integrated into a reprocessing system.

By way of example only, in the embodiment of FIGS. 1 and 2, the first fluid pathway 106 is incomplete and includes a gap in the region of the receptacle 115. As a result, the first fluid pathway 106 is incomplete when the cartridge 102 is not coupled to the adapter 104 (i.e., at least partially received in the receptacle 115). The gap can be filled, thereby completing the first fluid pathway 106, by at least a portion of the second fluid pathway 116 of the cartridge 102, when at least a portion of the cartridge 102 is received in the receptacle 115 of the adapter 104.

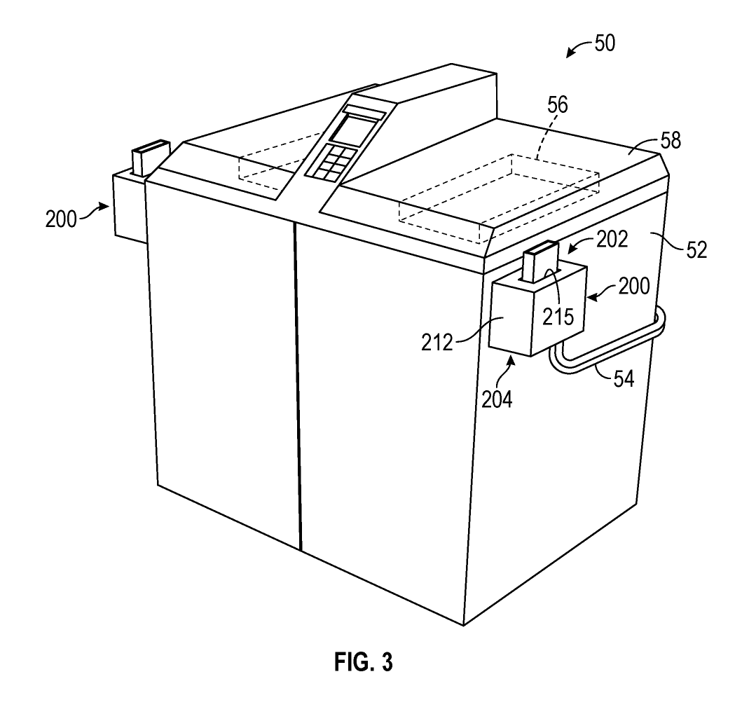

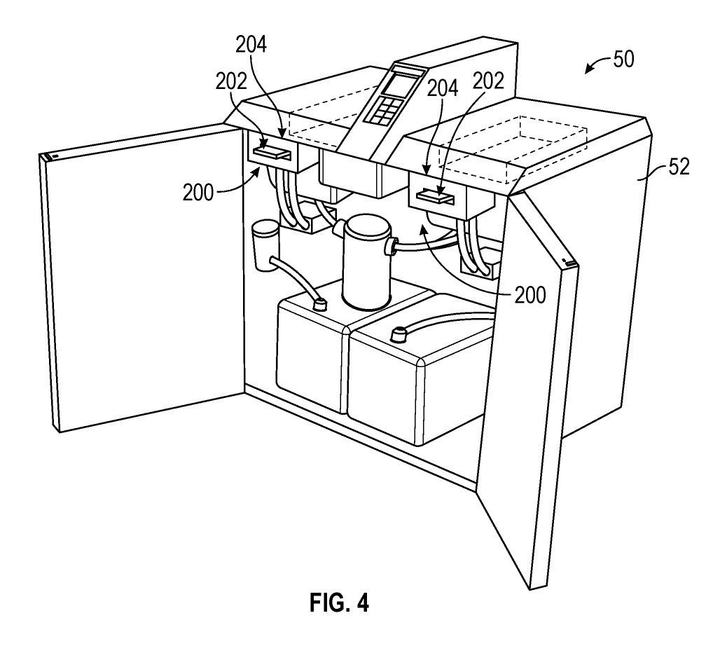

FIG. 3 illustrates an endoscope reprocessing system 50 according to one embodiment of the present disclosure. By way of example only, FIG. 3 is shown as including an automated endoscope reprocessor (AER). As shown, the endoscope reprocessing system 50 includes one or more process monitoring systems 200 according to another embodiment of the present disclosure. In the embodiment shown in FIG. 3, the endoscope reprocessing system 50 includes a housing 52 and includes two process monitoring systems 200.

Each process monitoring system 200 includes a cartridge 202 and an adapter 204. The adapter 204 includes a receptacle 215 dimensioned to receive at least a portion of the cartridge 202. As shown, the adapter 204 can further include a housing 212 that defines the receptacle 215.

By way of example, each process monitoring system 200 is shown in FIG. 3 as being located externally with respect to the housing 52 of the endoscope reprocessing system 50. In addition, as shown in FIG. 3, in some embodiments, one or more process monitoring systems 200 (and particularly, the adapter 204 thereof) can be coupled (e.g., removably coupled) to an outer surface of the housing 52.

The endoscope reprocessing system 50 can further include a fluid pathway 54 configured to be positioned in fluid communication with the process monitoring systems 200 and an endoscope. As a result, each of the process monitoring systems 200 can be fluidly coupled in series or in parallel with the endoscope. Furthermore, the endoscope reprocessing system 50 can include a basin 56 for receiving an endoscope (which can be covered by a portion of the housing 52, such as a cover 58). In such embodiments, the adapter 204 (or the process monitoring system 200 as a whole) can be located outside of the basin 56 (as shown in FIG. 3), inside the basin 56, or a combination thereof.

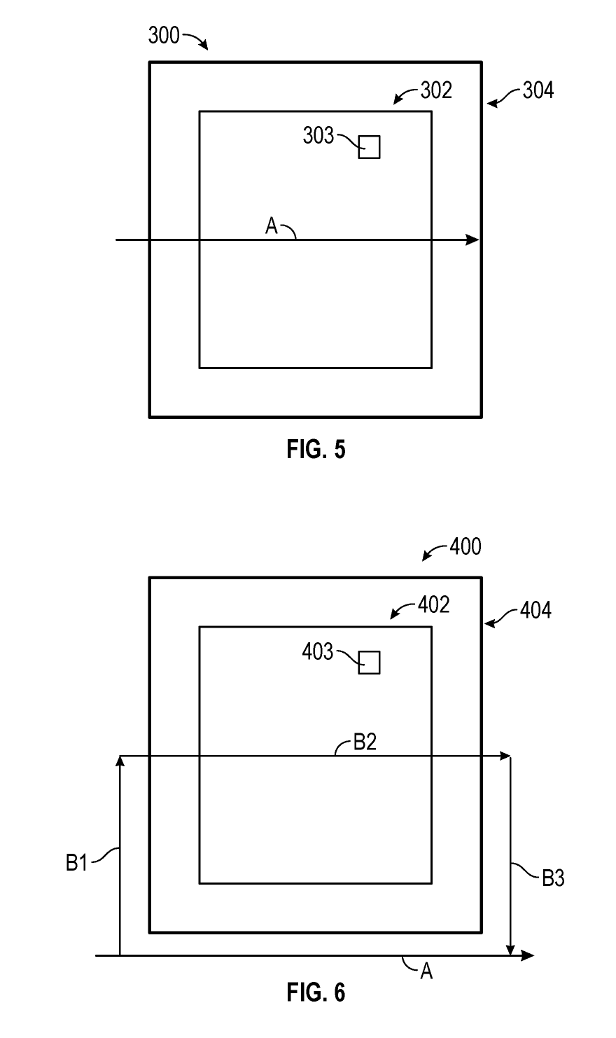

Additionally, or alternatively, in some embodiments, one or more process monitoring systems 200 can be located internally with respect to the housing 52, as shown in FIG. 4. In addition, as shown in FIG. 4, in some embodiments, one or more process monitoring systems 200 (and particularly, the adapter 204 thereof) can be coupled (e.g., removably coupled) to an inner surface of the housing 52. Now that the main components of process monitoring systems of the present disclosure have been introduced, the various possible configurations and additional features thereof will now be described in greater detail with reference to the schematic diagrams of FIGS. 5-15.

As mentioned above, in some embodiments, the process monitoring systems of the present disclosure can be connected to a reprocessing system, such that it is fluidly coupled to the other elements of the reprocessing system (e.g., a medical device to be reprocessed, such as an endoscope) in series, or in parallel. FIG. 5 schematically illustrates a process monitoring system 300 that includes a cartridge 302 comprising an indicator 303, and an adapter 304, and that is connected in series with a main fluid pathway through a reprocessing system, which is indicated by a bold arrow labeled A. By way of example, a medical device to be reprocessed can be located upstream or downstream of the process monitoring system 300, along the main fluid pathway A.

FIG. 6, on the other hand, schematically illustrates a process monitoring system 400 that includes a cartridge 402 comprising an indicator 403, and an adapter 404, and that is connected in parallel with a main fluid pathway through a reprocessing system, which is again indicated by a bold arrow labeled A. The diverted parallel fluid pathway through the process monitoring system 400 is indicated by a series of thinner arrows labeled as B1, B2 and B3, which form a circuit. By way of example, a medical device to be reprocessed can be located (e.g., fluidly coupled) anywhere along the main fluid pathway A. For example, in some embodiments, the medical device can be located between the locations where the arrow B1 exits the main fluid pathway A and where the arrow B3 re-enters the main fluid pathway A.

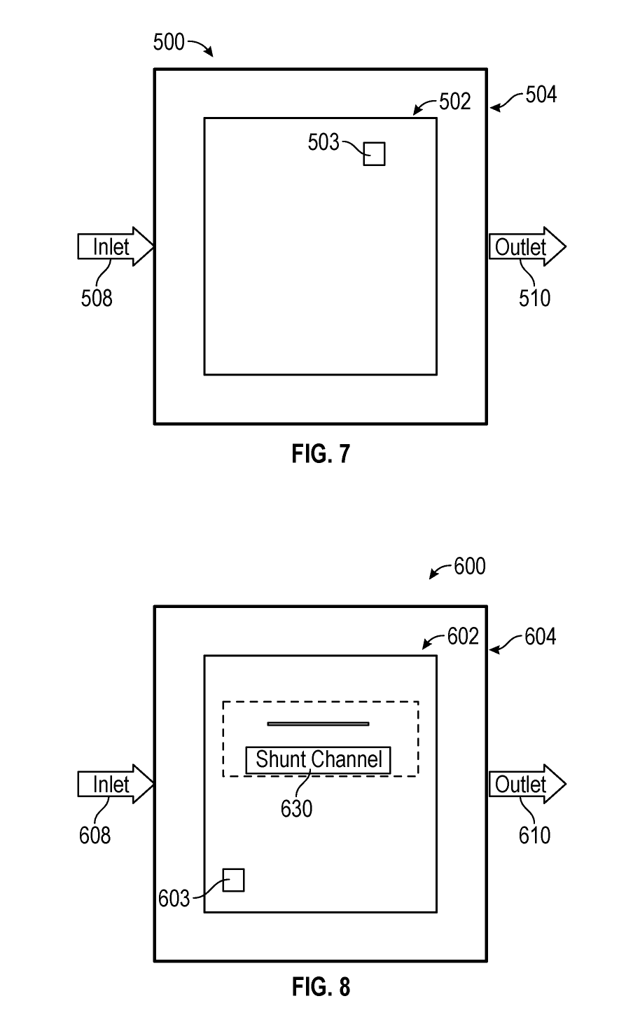

Specific optional features of process monitoring systems of the present disclosure will now be described with reference to FIGS. 7-15. FIGS. 7-15 illustrate various features and configurations of the cartridges, adapters and process monitoring systems of the present disclosure, wherein like numerals represent like elements. The cartridges, adapters and process monitoring systems of FIGS. 7-15 share many of the same elements, features, and functions as the process monitoring system 100 described above with respect to FIGS. 1-2. Reference is made to the description above accompanying FIGS. 1-2 for a more complete description of the features and elements (and alternatives to such features and elements) of the embodiments illustrated in FIGS. 7-15. Any of the features described above with respect to FIGS. 1-2 can be applied to the embodiments of FIGS. 7-15, and vice versa.

FIG. 7 shows a schematic representation of a process monitoring system 500 according to another embodiment of the present disclosure. The process monitoring system 500 includes a cartridge 502 and an adapter 504. Similar to the process monitoring system 100 of FIGS. 1 and 2, the adapter 504 includes a first fluid pathway that is configured to be positioned in fluid communication with a reprocessing system of interest.

In addition, the cartridge 502 includes a second fluid pathway configured to be positioned in fluid communication with the first fluid pathway of the adapter 504 when the cartridge 502 is received in a receptacle of the adapter 504 (represented schematically by the box element representing the cartridge 502 being located inside the box element representing the adapter 504). The cartridge 502 also includes an indicator 503.

The adapter 504 includes an inlet 508 (represented by an arrow) and outlet 510 (represented by an arrow) that allow for the first fluid pathway of the adapter 504 to be fluidly connected to the reprocessing system of interest, either in series or in parallel, as shown in FIGS. 5-6 and described above. As a result, when the process monitoring system 500 is connected to a reprocessing system, fluid from the reprocessing system can flow into the inlet 508, into the second fluid pathway of the cartridge 502, and out the outlet 510.

However, in the embodiment represented in FIG. 7, and similar to the embodiment of FIGS. 1-2, fluid does not flow through the process monitoring system 500 when a cartridge 502 is not coupled to the adapter 504. Said another way, the first fluid pathway in the adapter 504 can include a gap or be incomplete until a cartridge 502 is coupled to the adapter 504, thereby completing the first fluid pathway with the second fluid pathway of the cartridge 502. For such embodiments, it may be advantageous if the process monitoring system 500 is connected in parallel with other elements of the reprocessing system so as not to impede the overall fluid flow through the reprocessing system when the process monitoring system 500 is not fully assembled (i.e., when a cartridge 502 is not coupled to the adapter 504).

In some reprocessing systems, it may be important that the process monitoring system 500 does not significantly increase the resistance to fluid flow of the overall reprocessing system. In the embodiment of FIG. 7, there may be instances where the first fluid pathway of the adapter 504 and/or the second fluid pathway of the cartridge 502 may be formed of microfluidic channels, or at least channels of smaller dimensions than others of the reprocessing system, such that the resistance to fluid flow is substantially increased.

In some systems, this may not be an issue. However, in some reprocessing systems, it may be necessary that the process monitoring system provide fluid flow therethrough that is substantially the same as (or at least not greater than) the fluid flow through the rest of the reprocessing system.

For example, in some embodiments, the resistance to flow through a reprocessing system positioned in fluid communication with a process monitoring system of the present disclosure is increased due to the process monitoring system by no greater than 20%; in some embodiments, no greater than 15%; in some embodiments, no greater than 10%; and in some embodiments, no greater than 5%.

As a result, in some embodiments, as described below with respect to FIGS. 8, 9, 12, 13, 14 and 15, one or both of the cartridge and the adapter of a given process monitoring system can include a shunt channel A shunt channel of the present disclosure is generally a channel that allows fluid to flow through the process monitoring system (e.g., when no cartridge is coupled to the adapter and no monitoring is being performed) without introducing a significant increase in the resistance to fluid flow for the reprocessing system. That is, a shunt channel can be used to allow fluid to flow through the process monitoring system (e.g., via either the cartridge, the adapter, or both) in such a way that the resistance to fluid flow through the reprocessing system is not significantly impacted (or at least not significantly increased).

FIG. 8 shows a schematic representation of a process monitoring system 600 according to another embodiment of the present disclosure. The process monitoring system 600 includes a cartridge 602 (including an indicator 603), and an adapter 604 (with an inlet 608 and an outlet 610). The process monitoring system 600 includes all of the features of the process monitoring system 500 of FIG. 7, except that the process monitoring system 600 further includes a shunt channel 630.

By way of example only, the shunt channel 630 is shown as being located on the cartridge 602, and is in fluid communication with (or forms at least a portion of) the second fluid pathway on the cartridge 602. As a result, when the process monitoring system 600 is connected to a reprocessing system, fluid from the reprocessing system can flow into the inlet 608, into the second fluid pathway of the cartridge 602, and out the outlet 610. However, in the embodiment represented in FIG. 8, and similar to the embodiments of FIGS. 1-2 and 7, fluid does not flow through the process monitoring system 600 when a cartridge 602 is not coupled to the adapter 604. Said another way, the first fluid pathway in the adapter 604 can include a gap or be incomplete until a cartridge 602 is coupled to the adapter 604, thereby completing the first fluid pathway with the second fluid pathway of the cartridge 602. When the process monitoring system 600 is coupled to the reprocessing system (i.e., in fluid communication therewith) and the cartridge 602 is coupled to the adapter 604, fluid can flow through the reprocessing system via the shunt channel 630 without the process monitoring system 600 adding substantial resistance to the fluid flow in the reprocessing system.

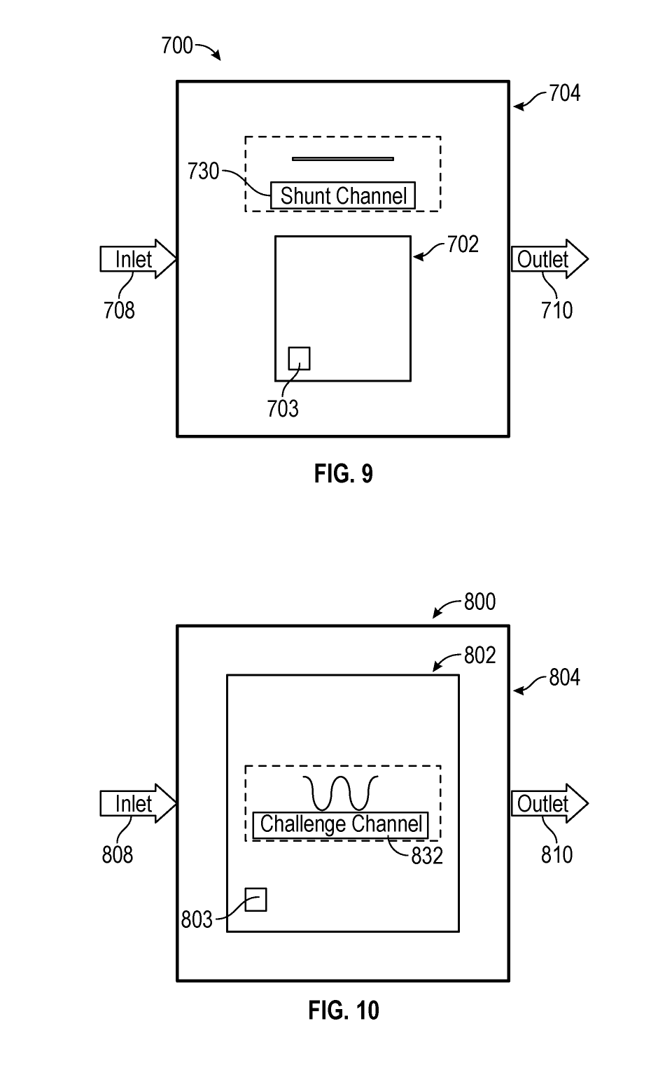

FIG. 9 shows a schematic representation of a process monitoring system 700 according to another embodiment of the present disclosure. The process monitoring system 700 includes a cartridge 702 (including an indicator 703), and an adapter 704 (with an inlet 708 and an outlet 710). The process monitoring system 700 includes all of the features of the process monitoring system 600 of FIG. 8, except that the process monitoring system 700 includes a shunt channel 730 that is located in the adapter 704, instead of the cartridge 702. That is, the shunt channel 730 is in fluid communication with (or forms at least a portion of) the first fluid pathway on the adapter 704. Thus, unlike earlier embodiments, the first fluid pathway of the adapter 704 is complete even when the cartridge 702 is not coupled to the adapter 704.

As a result, when the process monitoring system 700 is connected to a reprocessing system without a cartridge 702 being coupled to the adapter 704, fluid from the reprocessing system can flow into the inlet 708, into the first fluid pathway of the adapter 704, and out the outlet 710. In addition, because the first fluid pathway of the adapter 704 includes the shunt channel 730, fluid can flow through the reprocessing system via the shunt channel 730 without the process monitoring system 700 adding substantial resistance to the fluid flow in the reprocessing system. When a cartridge 702 is then coupled to the adapter 704, fluid can flow into the inlet 708, into the first fluid pathway, with a portion diverted (or sampled) to the second fluid pathway of the cartridge 702, such that the resistance to fluid flow through the reprocessing system is not substantially affected by the process monitoring system 700, because enough fluid is still being directed through the shunt channel 730 of the adapter 704.

As a result, when the process monitoring system 700 is connected to a reprocessing system without a cartridge 702 being coupled to the adapter 704, fluid from the reprocessing system can flow into the inlet 708, into the first fluid pathway of the adapter 704, and out the outlet 710. In addition, because the first fluid pathway of the adapter 704 includes the shunt channel 730, fluid can flow through the reprocessing system via the shunt channel 730 without the process monitoring system 700 adding substantial resistance to the fluid flow in the reprocessing system. When a cartridge 702 is then coupled to the adapter 704, fluid can flow into the inlet 708, into the first fluid pathway, with a portion diverted (or sampled) to the second fluid pathway of the cartridge 702, such that the resistance to fluid flow through the reprocessing system is not substantially affected by the process monitoring system 700, because enough fluid is still being directed through the shunt channel 730 of the adapter 704.

In some embodiments, the reprocessing system to be monitored by a process monitoring system of the present disclosure can include an endoscope reprocessing system. In such embodiments, it can be advantageous for the process monitoring system to be able to mimic the fluid flow through the endoscope by providing a challenge channel A challenge channel of the present disclosure is a channel or fluid pathway that is configured to mimic the challenge (e.g., process challenge) provided by an endoscope by providing a similar resistance to flow (e.g., volumetric fluid flow).

That is, a challenge channel of the present disclosure can be designed to mimic the resistance to flow (e.g., of a liquid, e.g., of a liquid disinfectant) in a flexible endoscope. Such a design can be based on Poiseuille's law. According to Poiseuille's law, in the case of laminar flow, the volumetric flowrate is given by the pressure difference divided by the viscous resistance. This resistance depends linearly upon the viscosity and the length, but the fourth power dependence upon the radius is dramatically different. Poiseuille's law is found to be in reasonable agreement with experiment for uniform liquids (Newtonian fluids) in cases where there is no appreciable turbulence.

According to Poiseuille's law, the volumetric flowrate is given by:

$$Volumetric Flowrate = ℱ = \frac{{P}_{1} - {P}_{2}}{ℛ} = \frac{π ( {P}_{1} - {P}_{2} ) {r}^{4}}{8 η L}$$

Where the resistance to flow [symbol] is given by:

$$ℛ = \frac{8 η L}{{π r}^{4}}$$

Where η the viscosity of the liquid (e.g., the liquid disinfect), L is the length of the channel, and r is the radius of the channel This relationship enables the design of a relatively small-footprint challenge channel (i.e., that is considerably condensed, relative to the life size of an endoscope) that mimics the challenge-to-flow posed to an endoscope reprocessing system by an endoscope having a long and narrow lumen.

For example, for a given liquid with a known viscosity, η, the resistance to flow, R, is proportional to L/r4. Thus, for a GI endoscope with a 5-mm lumen and a length of 2.5 m, L/r4 is 64 mm-3. To simulate a resistance that is equivalent to that of the 2.5-m endoscope using a challenge channel that is 2.5 mm in diameter, the length, L, of the challenge channel need only be 156 mm.

In some embodiments, however, the challenge channel may have a non-circular cross-sectional shape. In embodiments employing such a non-circular cross-sectional shape (such as square, rectangular, or annular channels, e.g., where the height and width are comparable), the characteristic dimension used to describe internal flow is the hydraulic radius, Rh, defined as:

$${R}_{h} = \frac{2 A}{P}$$

where A is the cross-sectional area and P is the wetted perimeter (i.e. the perimeter of the PCD challenge channel) of the challenge channel (Heat transfer, 10th ed., J. P. Holman, McGraw Hill, 2009).

Proper application of Poiseuille's law as the basis for describing the physics of flow in a challenge channel of the present disclosure assumes that flow within at least the challenge channel of the first and/or second fluid pathway of a process monitoring system of the present disclosure is laminar flow. This implies that the Reynolds number describing the fluid mechanics of the challenge channel is sufficiently low to exclude turbulent flow. Reynolds number Re is a dimensionless quantity defined as the ratio of inertial forces to viscous forces for given flow conditions. For flow in a channel, Re is defined as:

$${R}_{e} = \frac{2 \times {R}_{h} Q}{vA}$$

Rh is the hydraulic radius of the channel;
Q is the volumetric flow rate in the channel;
A is the cross sectional area of the channel; and
v is the kinematic viscosity of the fluid flowing through the channel

The onset of turbulent flow in a pipe or channel is generally taken to occur for Reynolds numbers >2300. In some embodiments, the Rₑ for challenge channels of the present disclosure can be less than 2300; in some embodiments, less than 2000; in some embodiments, less than 1500; in some embodiments, less than 1000; in some embodiments, less than 500; in some embodiments, less than 200; and in some embodiments, less than 150. As a result, flow in the challenge channels of the present disclosure can be considered laminar and application of Poiseuille's law is appropriate.

In some embodiments, the challenge channel can include one or more bends or turns or non-linear segments to reduce the overall footprint of the challenge channel and to fit a challenge channel have a greater length on a reduced area of a cartridge. However, as described above, the cross-sectional dimension (e.g., radius) and the length of the channel are the parameters that are controlled in order to create a challenge channel, and the challenge channels of the present disclosure need not include any bends, turns, or nonlinear segments.

In some embodiments, the challenge channels of the present disclosure can be configured to mimic only the resistance to flow for the working channel (also typically referred as the suction/biopsy channel) of a flexible endoscope, simplifying the challenge channel design to represent the resistance to flow of the working channel with respect to its length and diameter. In some embodiments, the challenge channels of the present disclosure can additionally mimic (or represent) additional channels that are also present in some of these devices (e.g., air/water channels, auxiliary water channels, elevator guide wire channels, or the like, or a combination thereof). In some embodiments, the challenge channels of the present disclosure can additionally mimic (or represent) the resistance to flow of additional fluidic components present in these devices (e.g., junctions, valves, dead flow volumes or retrograde channel flow). In addition, various types of endoscopes can be mimicked using a challenge channel of the present disclosure, including, but not limited to, gastroenterology endoscopes, bronchoscopes, laryngoscopes, hysteroscopes, intubation scopes, urology scopes, and other such devices.

FIG. 10 shows a schematic representation of a process monitoring system 800 according to another embodiment of the present disclosure. The process monitoring system 800 includes a cartridge 802 (including an indicator 803), and an adapter 804 (with an inlet 808 and an outlet 810). The process monitoring system 800 includes all of the features of the process monitoring system 500 of FIG. 7, except that the process monitoring system 800 further includes a challenge channel 832.

By way of example only, the challenge channel 832 is shown as being located on the cartridge 802, and is in fluid communication with (or forms at least a portion of) the second fluid pathway on the cartridge 802. As a result, when the process monitoring system 800 is connected to a reprocessing system, fluid from the reprocessing system can flow into the inlet 808, into the second fluid pathway of the cartridge 802, and out the outlet 810. However, in the embodiment represented in FIG. 10, and similar to the embodiments of FIGS. 1-2, 7, and 8, fluid does not flow through the process monitoring system 800 when a cartridge 802 is not coupled to the adapter 804. Said another way, the first fluid pathway in the adapter 804 can include a gap or be incomplete until a cartridge 802 is coupled to the adapter 804, thereby completing the first fluid pathway with the second fluid pathway of the cartridge 802. When the process monitoring system 800 is coupled to the reprocessing system (i.e., in fluid communication therewith) and the cartridge 802 is coupled to the adapter 804, fluid can flow through the reprocessing system via the challenge channel 832 to mimic the resistance to flow through an endoscope in the reprocessing system.

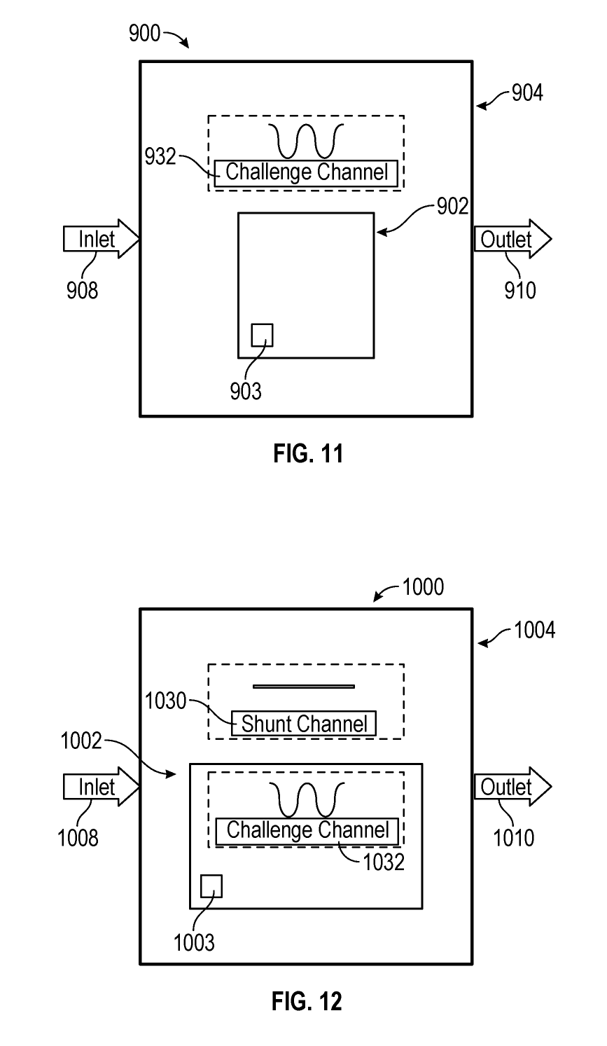

FIG. 11 shows a schematic representation of a process monitoring system 900 according to another embodiment of the present disclosure. The process monitoring system 900 includes a cartridge 902 (including an indicator 903), and an adapter 904 (with an inlet 908 and an outlet 910). The process monitoring system 900 includes all of the features of the process monitoring system 800 of FIG. 10, except that the process monitoring system 900 includes a challenge channel 932 that is located in the adapter 904, instead of the cartridge 902. That is, the challenge channel 932 is in fluid communication with (or forms at least a portion of) the first fluid pathway on the adapter 904. As a result, in some embodiments, the first fluid pathway of the adapter 904 can be complete even when the cartridge 902 is not coupled to the adapter 904, and itself can mimic the resistance to flow through an endoscope in the reprocessing system.

As a result, when the process monitoring system 900 is connected to a reprocessing system without a cartridge 902 being coupled to the adapter 904, fluid from the reprocessing system can flow into the inlet 908, into the first fluid pathway of the adapter 904, and out the outlet 910. In addition, because the first fluid pathway of the adapter 904 includes the challenge channel 932, fluid can flow through the reprocessing system via the challenge channel 932 even when a cartridge 902 is not present. When a cartridge 902 is then coupled to the adapter 904, fluid can flow into the inlet 908, into the first fluid pathway which includes the challenge channel 932, with a portion diverted (or sampled) to the second fluid pathway of the cartridge 902, such that the resistance to fluid flow through the reprocessing system mimics that of an endoscope, while a portion of the fluid is still sampled (i.e., tested) by the cartridge 902.

FIG. 12 shows a schematic representation of a process monitoring system 1000 according to another embodiment of the present disclosure. The process monitoring system 1000 includes a cartridge 1002 (including an indicator 1003), and an adapter 1004 (with an inlet 1008 and an outlet 1010). The process monitoring system 1000 includes all of the features of the process monitoring system 800 of FIG. 10, except that the process monitoring system 1000 further includes a shunt channel 1030. That is, in the process monitoring system 1000, the cartridge 1002 includes a challenge channel 1032 in fluid communication with (or forming a portion of) the second fluid pathway, and the adapter 1004 includes the shunt channel 1030 in fluid communication with (or forming a portion of) the first fluid pathway.

As a result, the process monitoring system 1000 is a combination of the process monitoring system 700 of FIG. 9 and the process monitoring system 800 of FIG. 10. Accordingly, because the first fluid pathway of the adapter 1004 includes the shunt channel 1030, fluid can flow through the reprocessing system via the shunt channel 1030 without the process monitoring system 1000 adding substantial resistance to the fluid flow in the reprocessing system. When a cartridge 1002 is then coupled to the adapter 1004, fluid can flow into the inlet 1008, into the first fluid pathway, with a portion diverted (or sampled) to the second fluid pathway of the cartridge 1002, such that the resistance to fluid flow through the reprocessing system is not substantially affected by the process monitoring system 1000, because enough fluid is still being directed through the shunt channel 1030 of the adapter 1004. That is, when the cartridge 1002 is coupled to the adapter 1004, the challenge channel 1032 is fluidly coupled to the shunt channel 1030 in parallel.

In addition, the challenge channel 1032 is located on the cartridge 1002, such that when the cartridge 1002 is coupled to the adapter 1004, fluid can flow through the second fluid pathway (or be sampled into the second fluid pathway) via the challenge channel 1032 to mimic the resistance to flow through an endoscope in the reprocessing system.

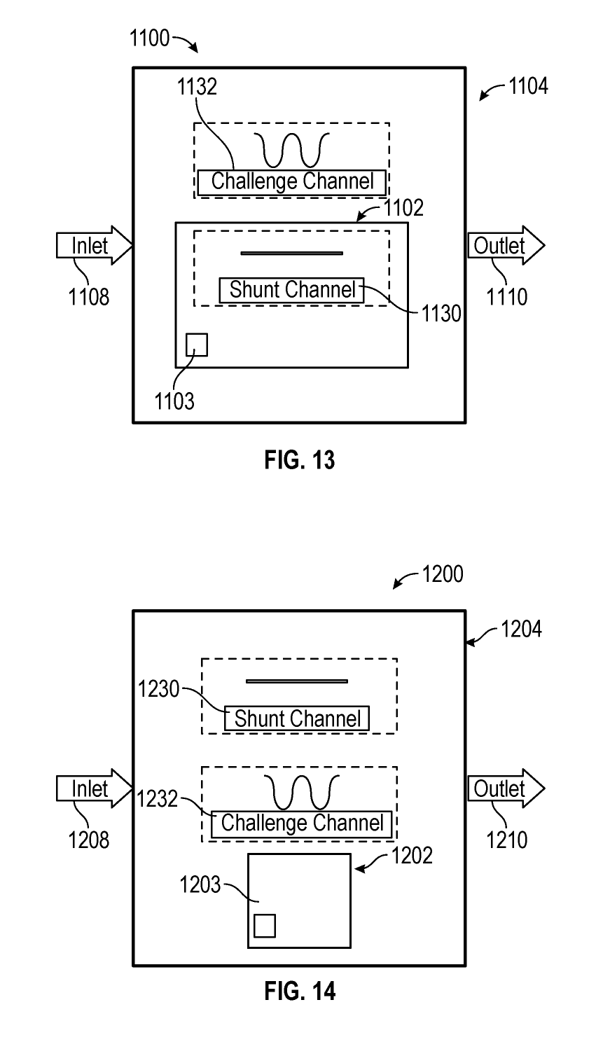

FIG. 13 shows a schematic representation of a process monitoring system 1100 according to another embodiment of the present disclosure. The process monitoring system 1100 includes a cartridge 1102 (including an indicator 1103), and an adapter 1104 (with an inlet 1108 and an outlet 1110). The process monitoring system 1100 includes all of the features of the process monitoring system 1000 of FIG. 12, except that in the process monitoring system 1100, a shunt channel 1130 is located on the cartridge 1102 (i.e., is in fluid communication with, or forms a portion of, the second fluid pathway), and the challenge channel 1132 is located on the adapter 1104 (i.e., is in fluid communication with, or forms a portion of, the first fluid pathway).

As a result, the process monitoring system 1100 is a combination of the process monitoring system 600 of FIG. 8 and the process monitoring system 900 of FIG. 11. Accordingly, when the process monitoring system 1100 is coupled to the reprocessing system (i.e., in fluid communication therewith) and the cartridge 1102 is coupled to the adapter 1104, fluid can flow through the reprocessing system via the shunt channel 1130 on the cartridge 1102 without the process monitoring system 1100 adding substantial resistance to the fluid flow in the reprocessing system.

In addition, the challenge channel 1132 is located on the adapter 1104, such that even when the cartridge 1102 is not coupled to the adapter 1104, fluid can flow through the challenge channel 1132 to mimic the resistance to flow through an endoscope in the reprocessing system. However, in such embodiments, a cartridge 1102, which includes the shunt channel 1130, can be coupled to the adapter 1104 to allow a substantial portion of the fluid to flow through the shunt channel 1130, such that the process monitoring system 1100 does not significantly increase the resistance to flow of the reprocessing system.

FIG. 14 shows a schematic representation of a process monitoring system 1200 according to another embodiment of the present disclosure. The process monitoring system 1200 includes a cartridge 1202 (including an indicator 1203), and an adapter 1204 (with an inlet 1208, an outlet 1210 and a shunt channel 1230). The process monitoring system 1200 includes all of the features of the process monitoring system 1000 of FIG. 12, except that in the process monitoring system 1200, a challenge channel 1232 is also located on the adapter 1204 (i.e., is in fluid communication with, or forms a portion of, the first fluid pathway). In such embodiments, the cartridge 1202 can still be configured to be received in a receptacle of the adapter 1204 to position the second fluid pathway of the cartridge 1202 in fluid communication with the first fluid pathway of the adapter 1204, but the adapter 1204 includes the lower resistance channel, the shunt channel 1230, and the higher resistance channel, the challenge channel 1232.

In some embodiments, it can be advantageous for the shunt channel 1230 and the challenge channel 1232 to still be fluidly connected in parallel, such that a portion of the fluid flow through the first fluid pathway is sampled into the higher resistance challenge channel 1232 without significantly increasing the overall resistance to flow (i.e., by allowing a majority of the fluid to flow unaffected through the shunt channel 1230). As a result, because the first fluid pathway of the adapter 1204 includes the shunt channel 1230, fluid can flow through the reprocessing system via the shunt channel 1230 without the process monitoring system 1200 adding substantial resistance to the fluid flow in the reprocessing system.

Furthermore, because both the shunt channel 1230 and the challenge channel 1232 are located on the adapter 1204, fluid can flow through the adapter 1204 even when a cartridge 1202 is not coupled to the adapter 1204. Thus, the first fluid pathway of the adapter 1204 is complete even when the cartridge 1202 is not coupled to the adapter 1204, and itself mimics the resistance to flow through an endoscope in the reprocessing system. As a result, when the process monitoring system 1200 is connected to a reprocessing system without a cartridge 1202 being coupled to the adapter 1204, fluid from the reprocessing system can flow into the inlet 1208, into the first fluid pathway of the adapter 1204, and out the outlet 1210.

In addition, because the first fluid pathway of the adapter 1204 includes the challenge channel 1232, fluid can flow through the reprocessing system via the challenge channel 1232 even when a cartridge 1202 is not present. When a cartridge 1202 is then coupled to the adapter 1204, fluid can flow into the inlet 1208, into the first fluid pathway which includes the shunt channel 1230 and the challenge channel 1232, with a portion diverted (or sampled) to the second fluid pathway of the cartridge 1202 for contact with the indicator 1203, such that the resistance to fluid flow through the reprocessing system mimics that of an endoscope, while a portion of the fluid is still sampled (i.e., tested) by the cartridge 1202.

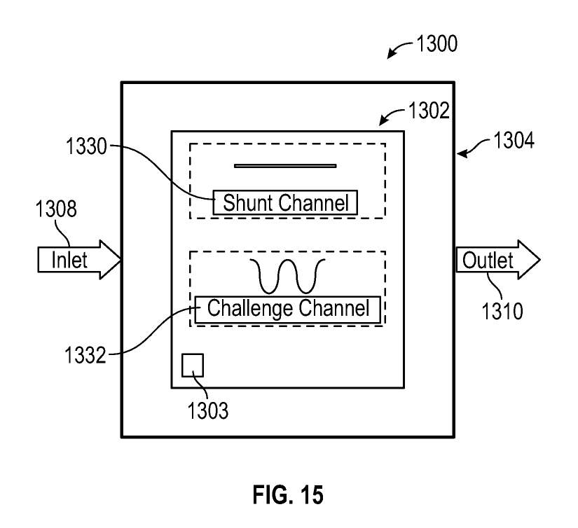

FIG. 15 shows a schematic representation of a process monitoring system 1300 according to another embodiment of the present disclosure. The process monitoring system 1300 includes a cartridge 1302 (including an indicator 1303), and an adapter 1304 (with an inlet 1308 and an outlet 1310). The process monitoring system 1300 includes all of the features of the process monitoring system 1200 of FIG. 14, except that the process monitoring system 1300 includes a shunt channel 1330 and a challenge channel 1332 that are both located on the cartridge 1302, i.e., in fluid communication with (or forming at least a portion of) the second fluid pathway on the cartridge 1302.

By way of example only, the shunt channel 1330 and the challenge channel 13325 are shown as being located on the cartridge 1302, and in fluid communication with (or forming at least a portion of) the second fluid pathway on the cartridge 1302. As a result, when the process monitoring system 1300 is connected to a reprocessing system, with the cartridge 1302 coupled to the adapter 1304, fluid from the reprocessing system can flow into the inlet 1308, into the second fluid pathway of the cartridge 1302, and out the outlet 1310. However, fluid does not flow through the process monitoring system 1300 when a cartridge 1302 is not coupled to the adapter 1304. Said another way, the first fluid pathway in the adapter 1304 can include a gap or be incomplete until a cartridge 1302 is coupled to the adapter 1304, thereby completing the first fluid pathway with the second fluid pathway of the cartridge 1302. When the process monitoring system 1300 is coupled to the reprocessing system (i.e., in fluid communication therewith) and the cartridge 1302 is coupled to the adapter 1304, fluid can flow through the reprocessing system via the shunt channel 1330 and the challenge channel 1332 (which can be connected in parallel) without the process monitoring system 1300 adding substantial resistance to the fluid flow in the reprocessing system while also using the challenge channel 1332 to mimic the resistance to flow through an endoscope in the reprocessing system.

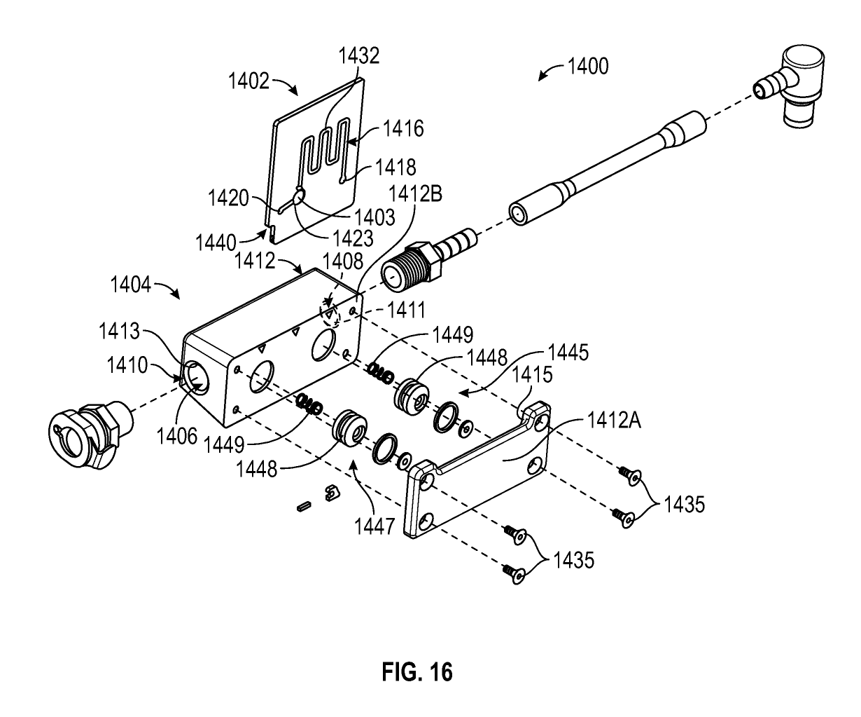

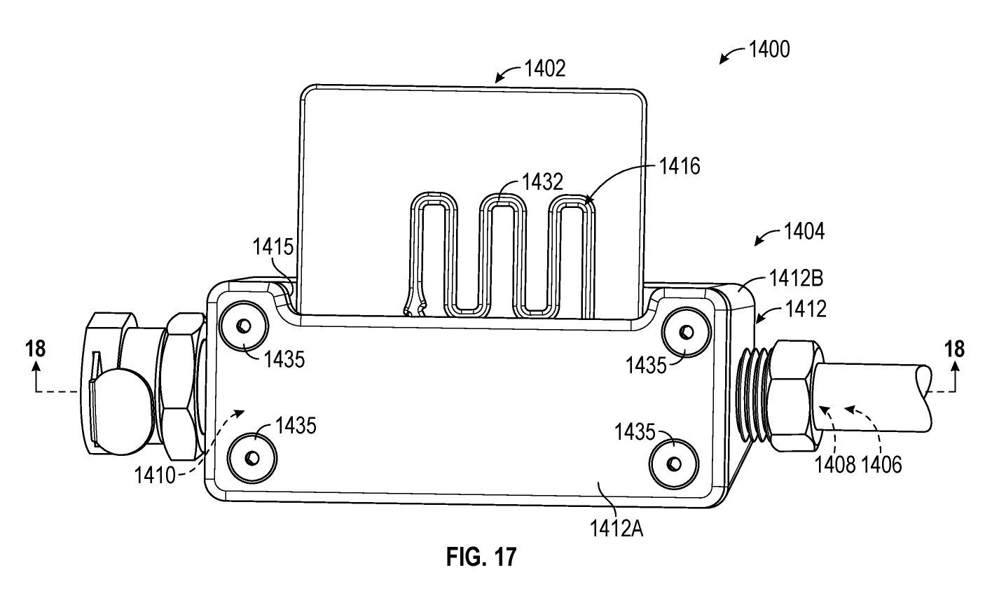

Specific structural embodiments of process monitoring systems of the present disclosure will now be described with reference to FIGS. 16-23. FIGS. 16-23 illustrate various features and configurations of the cartridges, adapters and process monitoring systems of the present disclosure, wherein like numerals represent like elements. The cartridges, adapters and process monitoring systems of FIGS. 16-23 share many of the same elements, features, and functions as the process monitoring systems described above with respect to FIGS. 1-15. Reference is made to the description above accompanying FIGS. 1-15 for a more complete description of the features and elements (and alternatives to such features and elements) of the embodiments illustrated in FIGS. 16-23. Any of the features described above with respect to FIGS. 1-15 can be applied to the embodiments of FIGS. 16-23, and vice versa.

FIGS. 16-18 illustrate a process monitoring system 1400 according to one embodiment of the present disclosure, including a cartridge 1402 according to one embodiment of the present disclosure, and an adapter 1404 according to one embodiment of the present disclosure. The cartridge 1402 can include one or more indicators 1403 configured to indicate a monitoring result (e.g., a pass or fail) to a user regarding whether a particular reprocessing system or process of interest met the necessary conditions. As shown in FIGS. 16-18, the adapter 1404 includes a first fluid pathway 1406 that is open-ended (i.e., when not coupled to a reprocessing system), including an inlet 1408 and an outlet 1410. The first fluid pathway 1406 can be configured to be positioned in fluid communication with a reprocessing system via the inlet 1408 and the outlet 1410, and optionally via one or more connectors and tubings, as shown. Such tubing can be flexible which can allow the adapter 1404 to be more easily positioned in a physical space desired (e.g., in an AER basin). In addition, by employing particular connectors with the process monitoring system 1400, the process monitoring system 1400 (and specifically, the adapter 1404) can be coupled directly to a reprocessing instrument or structure (e.g., a commercially available connection harness for an AER) directly without requiring modification of the instrument or structure. As mentioned above, the process monitoring system 1400 can be fluidly coupled to a reprocessing system of interest in series or in parallel to the fluid flow through a medical device (e.g., an endoscope) to be reprocessed.

The adapter 1404 can further include a housing 1412 that can include and define at least a portion of the first fluid pathway 1406. For example, as shown in FIG. 16, the housing 1412 can include a first opening 1411 that can define at least a portion of the inlet 1408 of the first fluid pathway 1406, and a second opening 1413 that can define at least a portion of the outlet 1410 of the first fluid pathway 1406. In some embodiments, the inlet 1408 and/or the outlet 1410 can be formed not only by the adapter housing 1412 itself, but possibly by other connectors or fittings coupled to the housing 1412 that themselves define at least a portion of the first fluid pathway 1406 (e.g., the inlet 1408 and/or the outlet 1410).

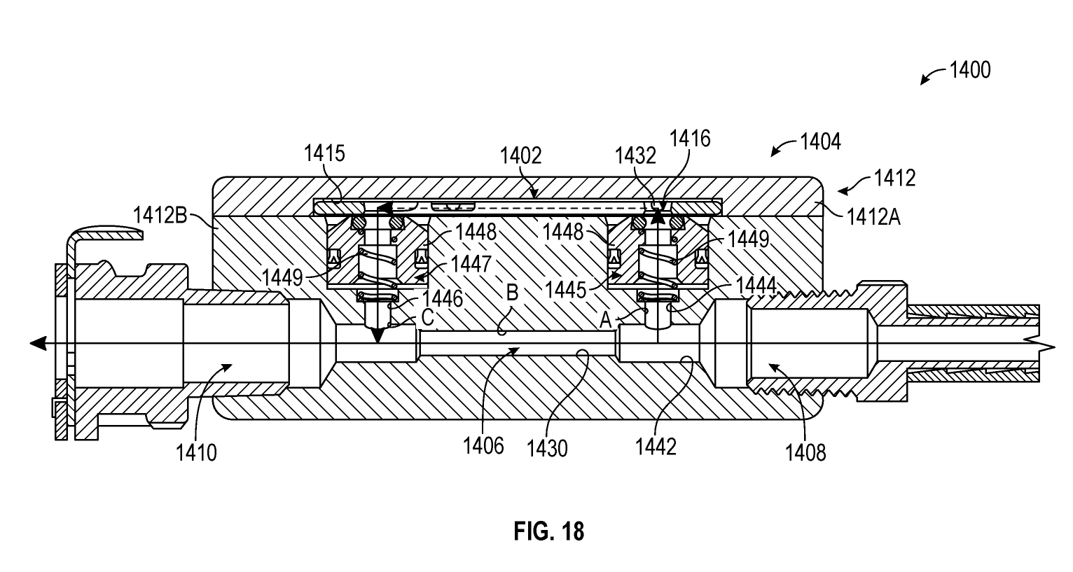

In some embodiments, the housing 1412 can be formed of more than one portion that are configured to be coupled together (e.g., permanently or removably). As further shown in FIGS. 16 and 18 by way of example only, the housing 1412 include a first portion 1412A (e.g., a face plate) that can be coupled to a second portion 1412B, e.g., by one or more fasteners 1435 (four screws are shown in FIGS. 16 and 17 by way of example only). By way of further example, the second portion 1412B of the housing 1412 forms the main body of the adapter 1404, housing the first fluid pathway 1406.

As further shown in FIGS. 16-18, the housing 1412 can further include a receptacle 1415 dimensioned to receive at least a portion of the cartridge 1402. By way of example only, the receptacle 1415 is formed between the first portion 1412A and the second portion 1412B of the adapter housing 1412.

By way of example only, in the embodiment of FIGS. 16-18, the receptacle 1415 is in the form of an open slot, and the cartridge 1402 is a flat, thin card that is shaped and sized such that at least a portion of the card can be slid into the receptacle 1415. However, it should be understood that other receptacle and card coupling configurations can be employed, as mentioned above with respect to FIGS. 1 and 2. In some embodiments, the cartridge 1402 can have a thickness that is no greater than 25 mm; in some embodiments, no greater than 20 mm; in some embodiments, no greater than 15 mm; in some embodiments, no greater than 10 mm; in some embodiments, no greater than 8 mm; in some embodiments, no greater than 5 mm; in some embodiments, no greater than 3 mm; in some embodiments, no greater than 2 mm; and in some embodiments, no greater than 1 mm.

As mentioned above, in some embodiments, the cartridge 1402 can be received in the receptacle 1415 with an audible and/or tactile feedback to the user that informs the user that the cartridge 1402 has been fully and properly positioned in the receptacle 1415. In addition, as shown in FIG. 16, in some embodiments, the cartridge 1402 can include one or more orientation features 1440, such that the cartridge 1402 is keyed with respect to the receptacle 1415 so as to permit positioning the cartridge 1402 in the receptacle 1415 in only one orientation.

The cartridge 1402 can include a second fluid pathway 1416 that is open-ended (i.e., when the cartridge 1402 is not coupled to the adapter 1404), includes an inlet 1418 and an outlet 1420 (see FIG. 16), and is configured to be positioned in fluid communication with the first fluid pathway 1406 of the adapter 1404 when at least a portion of the cartridge 1402 is received in the receptacle 1415 of the adapter 1404. As a result, the second fluid pathway 1416 of the cartridge 1402 can be in fluid communication with (e.g., can form a portion of) the first fluid pathway 1406 of the adapter 1404 when the cartridge 1402 is at least partially received in the receptacle 1415 of the adapter 1404, and fluid flow through the first fluid pathway 1406 of the adapter 1404 can be at least partially diverted through, or moved through, the second fluid pathway 1416 of the cartridge 1402. In addition, at least a portion of fluid flow in the reprocessing system into which the process monitoring system 1400 is incorporated (e.g., from a pump to an endoscope) can be routed through the cartridge 1402, e.g., before entering the endoscope. The portion of fluid flow that is sampled by the cartridge 1402 can vary. By way of example only, the process monitoring system 1400 has the same configuration as the process monitoring system 1000 of FIG. 12, in which the adapter 1404 includes a shunt channel 1430 (see FIG. 18), and the cartridge 1402 includes a challenge channel 1432. The challenge channel 1432 is configured to mimic the resistance to flow of a medical device of interest, e.g., an endoscope, as described above.

In addition, in some embodiments, as shown in FIGS. 16-18, when at least a portion of the cartridge 1402 is received in the receptacle 1415 of the adapter 1404, the second fluid pathway 1416 of the cartridge 1402 can be fluidly coupled to the first fluid pathway 1406 such that the inlet 1418 and the outlet 1420 of the second fluid pathway 1416 of the cartridge 1402 are located between the inlet 1408 and the outlet 1410 of the first fluid pathway 1406 of the adapter 1404, and particularly, between the first and second openings 1411 and 1413 in the housing 1412. The cartridge 1402 can be movable with respect to the adapter 1404 between (i) a first position (see FIG. 16) in which the cartridge 1402 is not received in the receptacle 1415 of the adapter 1404, and the second fluid pathway 1416 is not in fluid communication with the first fluid pathway 1406 of the adapter 1404; and (ii) a second position (see FIGS. 17 and 18) in which the cartridge 1402 is at least partially received in the receptacle 1415 of the adapter 1404, and the second fluid pathway 1416 is in fluid communication with the first fluid pathway 1406 of the adapter 1404.

A person of ordinary skill in the art will appreciate that the first and second fluid pathways 1406, 1416 can include gaskets, seals and/or valves (e.g., at the inlets 1408, 1418 and outlets 1410, 1420) to control the fluid connection between the first and second fluid pathways 1406, 1416 and to prevent leaks.

As shown in FIG. 16, the cartridge 1402 can include one or more indicators 14031ocated on the cartridge 1402, which can include at least one of a chemical indicator and a biological indicator, as described above. One indicator 1403 is shown in FIG. 16 by way of example only. As further shown in FIG. 16, the indicator 1403 can be located in a chamber 1423 that is in fluid communication with the second fluid pathway 1416, and particularly, with the challenge channel 1432, such that fluid moving through the second fluid pathway 1416 will contact the indicator 1403. In some embodiments, the cartridge 1402 can be transparent or can include a transparent window adjacent the chamber 1423, such that the indicator 1403 is visible or detectable by a reading apparatus, e.g., after being removed from being coupled to the adapter 1404.

The cartridge 1402 can form a removable and consumable portion of the process monitoring system 1400, and the adapter 1404 can form a more permanent portion of the process monitoring system 1400. In some embodiments, the adapter 1404 can be integrated into a reprocessing system.

As mentioned above, the adapter 1404 includes the shunt channel 1430, and a portion of the fluid flow through the adapter 1404 can be diverted (e.g., sampled) to the cartridge 1402 for monitoring. Because the shunt channel 1430 is configured not to impede fluid flow in a main fluid pathway of a reprocessing system to which the process monitoring system 1400 is coupled, and the challenge channel 1432 is positioned in parallel with the shunt channel 1430, the process monitoring system 1400 does not significantly affect, i.e., increase, the resistance to fluid flow of the reprocessing system. In addition, when a cartridge 1402 is not coupled to the adapter 1404, fluid can flow through the adapter 1404 without being sampled, again, not affecting the overall resistance to flow of the reprocessing system.

Fluid flow through the process monitoring system 1400 will now be described in greater detail with reference to FIG. 18. Fluid flow through the process monitoring system 1400 is generally shown with bold arrows, with a dashed arrow schematically representing the fluid flow through the cartridge 1402, and particularly, the challenge channel 1432 and the chamber 1423 where the indicator 1403 is located. The shunt channel 1430 is sized so as to provide no additional resistance to flow to a reprocessor, such as an AER, e.g., than a native hookup configuration. Located along the first fluid pathway 1406 are diversion ports to and from the cartridge 1402. As a result, in some embodiments, the first fluid pathway 1406 can be described as having (i) a primary path 1442, (ii) a first secondary path 144 positioned to connect the primary path 1442 to the inlet 1418 (see FIG. 16) of the second fluid pathway 1416 of the cartridge 1402, e.g., when at least a portion of the cartridge 1402 is received in the receptacle 1415 of the adapter 1404, and (iii) a second secondary path 1446 positioned to connect the primary path 1442 to the outlet 1420 (see FIG. 16) of the second fluid pathway 1416, e.g., when at least a portion of the cartridge 1402 is received in the receptacle 1415 of the adapter 1404. Flow from the primary path 1442 through the second fluid pathway 1416 of the cartridge 1402, e.g., via the first and second secondary paths 1444 and 1446, can be achieved in a variety of ways. By way of example only, flow is generated into and out of the second fluid pathway 1416 using the Venturi Effect (Bernoulli Principle). This can have the advantage of creating little to no additional resistance to a reprocessor (e.g., to an AER pump), which can be an important consideration in some cases.

As shown in FIG. 18, fluid can enter the adapter 1404 into a larger chamber, thus causing the fluid to decelerate. As the fluid enters the center cross-hole of the primary path 1442, it accelerates back to the same velocity it was in the tubing feeding the adapter 1404 (i.e., it has the same internal diameter). Finally, as the fluid exits the adapter 1404, it goes into another larger cross-sectional area, where it again decelerates. This sets up a lower pressure at the outlet port from the cartridge 1402, i.e., at the second secondary path 1446, and causes fluid to flow through the cartridge 1402 (i.e., when the cartridge 1402 is coupled to the adapter 1404). The pressure differential is used between the locations at which the first and second secondary paths 1444 and 1446 connect with the primary path 1442 (i.e., at positions marked “A” and “C” in FIG. 18), which provides a relatively low flow through the cartridge 1402. For higher flows, the second secondary path 1446 could connect at a position marked “B” in FIG. 18, where the highest pressure differential exists. As a result, in some embodiments, the primary path 1442 can include a venturi located upstream of the first secondary path 1444 and/or a venturi located downstream of the second secondary path 1446.

In some embodiments, the process monitoring system 1400 can further include a first valve 1445 (see FIGS. 16 and 18) positioned in the first secondary path 1444 and a second valve 1447 positioned in the second secondary path 1446 to inhibit fluid flow away from the primary path 1442 when the cartridge 1402 is not received in the receptacle 1415 of the adapter 1404 and to prevent leaks.

By way of example, in some embodiments, the first and second valves 1445 and 1447 can each include a plunger mechanism that can be used to open and close the first and second secondary paths 1444 and 1446 to and from the cartridge 1402. One or more plungers 1448 (see FIGS. 16 and 18) and a corresponding one or more springs 1449 can be positioned in each of the secondary paths 1444, 1446. One plunger mechanism will be described for simplicity and clarity. When the cartridge 1402 is not coupled to the adapter 1404, the plunger 1448 can push a seal (e.g., a cone seals), which can be coupled to or form a portion of the plunger 1448, against the first portion 1412A of the housing 1412 of the adapter 1404, ensuring that no fluid leaks out.

When a properly oriented cartridge 1402 is coupled to the adapter 1404, the spring 1449 can be compressed, allowing the cartridge 1402 to be moved (e.g., slid) from its first position to its second position between the first and second portions 1412A and 1412B of the housing 1412. When the cartridge 1402 is fully seated, the seal of the plunger 1448 is positioned right over the inlet 1418 or the outlet 1420 of the cartridge 1402, providing a fluidic connection. In this state, a portion of the fluid flowing through the adapter 1404 can enter the cartridge 1402 and flow through the challenge channel 1432 and the indicator chamber 1423, to ultimately exit the cartridge 1402, reentering the primary path 1442 of the adapter 1404. In embodiments in which the process monitoring system 1400 is used with an AER, all of the disinfectant exiting the outlet port in the AER basin will end up flowing into the endoscope, in other words, using the adapter 1404 prevents diverting any of the disinfectant away from the endoscope.

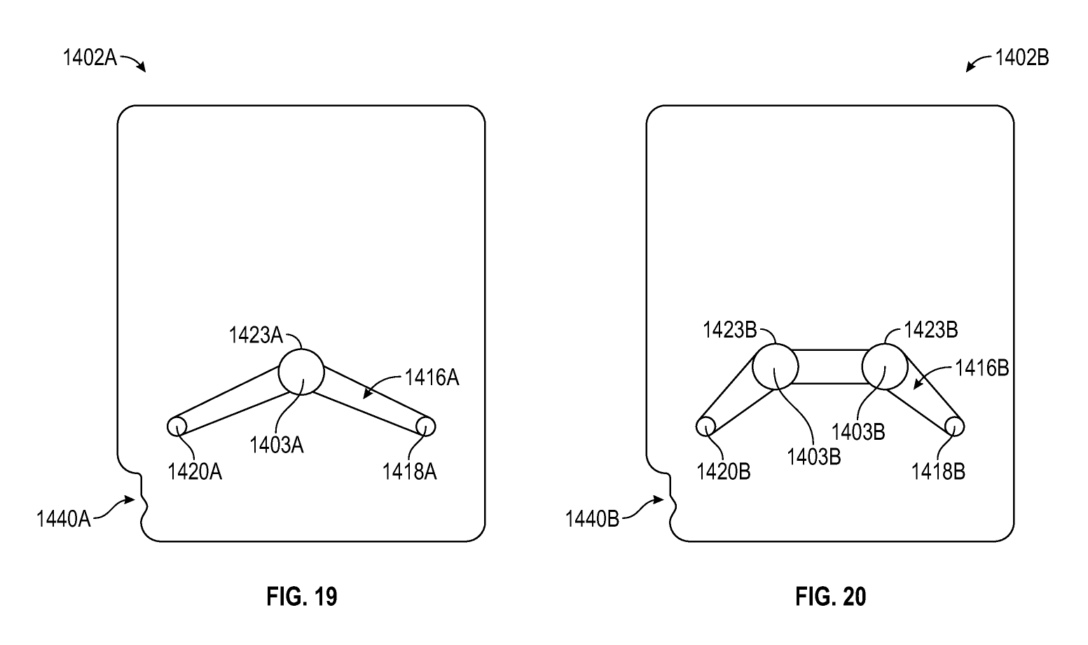

FIGS. 19 and 20 each illustrate an alternative cartridge configuration that can be employed with the process monitoring system 1400 of FIGS. 16-18. FIG. 19 illustrates a cartridge 1402A according to another embodiment of the present disclosure, and FIG. 20 illustrates a cartridge 1402B according to another embodiment of the present disclosure. The cartridge 1402A of FIG. 19 includes a second fluid pathway 1416A having an inlet 1418A and an outlet 1420A, with an indicator chamber 1423A positioned in fluid communication therewith and housing an indicator 1403A. The cartridge 1402A further includes an orientation feature 1440A configured to be keyed with respect to the adapter 1404 of FIGS. 16-18. By way of example only, the cartridge 1402A is a relatively simple example of a cartridge of the present disclosure, with only one indicator 1403, and where the second fluid pathway 1416A does not includes a challenge channel, and likely also not a shunt channel (similar to the embodiments of FIGS. 7, 9, 11 and 14 described above).

The cartridge 1402B of FIG. 20 includes a second fluid pathway 1416B having an inlet 1418B and an outlet 1420B, with two indicator chambers 1423B positioned in fluid communication therewith and each housing an indicator 1403B. The cartridge 1402 further includes an orientation feature 1440B configured to be keyed with respect to the adapter 1404 of FIGS. 16-18. That is, the cartridge 1402B is substantially the same as the cartridge 1402A of FIG. 19, except that the cartridge 1402B includes two chambers 1423B and two indicators 1403B. The two indicators 1403B can include two chemical indicators, two biological indicators, or one of each.

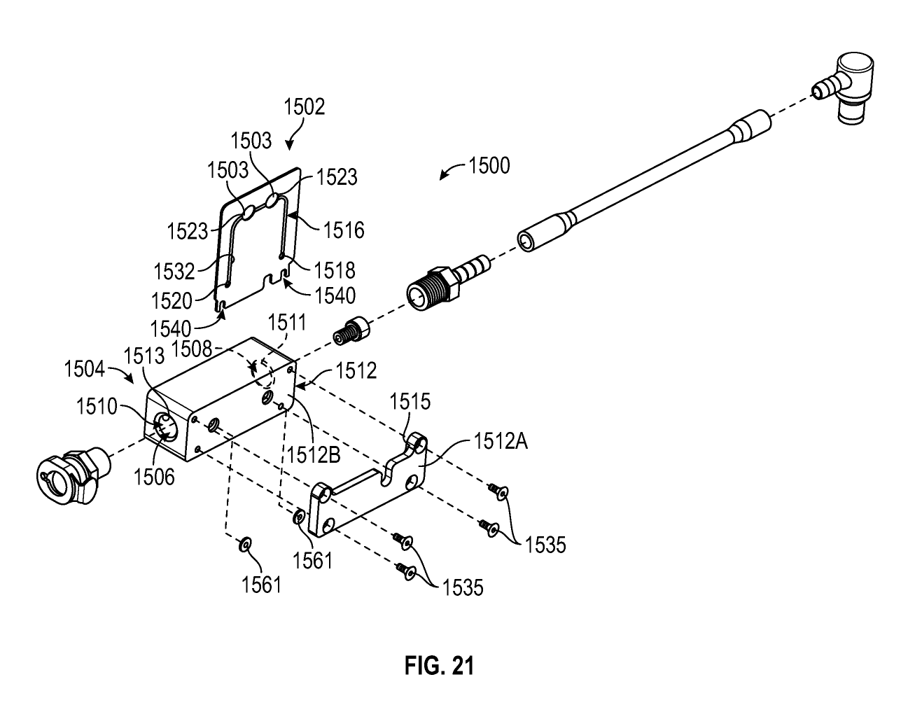

FIGS. 21-23 illustrate a process monitoring system 1500 according to one embodiment of the present disclosure, including a cartridge 1502 according to one embodiment of the present disclosure, and an adapter 1504 according to one embodiment of the present disclosure. The cartridge 1502 can include one or more indicators 1503. The process monitoring system 1500 is similar to the process monitoring system 1400 of FIGS. 16-18, except for a different adapter housing configuration, primary path configuration through the adapter 1504, and a different cartridge configuration 1502. As a result, reference is made to the description above accompanying FIGS. 16-18 for a more complete description of the features and elements (and alternatives to such features and elements) of the embodiments illustrated in FIGS. 21-23. Any of the features described above with respect to FIGS. 16-18 can be applied to the embodiments of FIGS. 21-23, and vice versa.

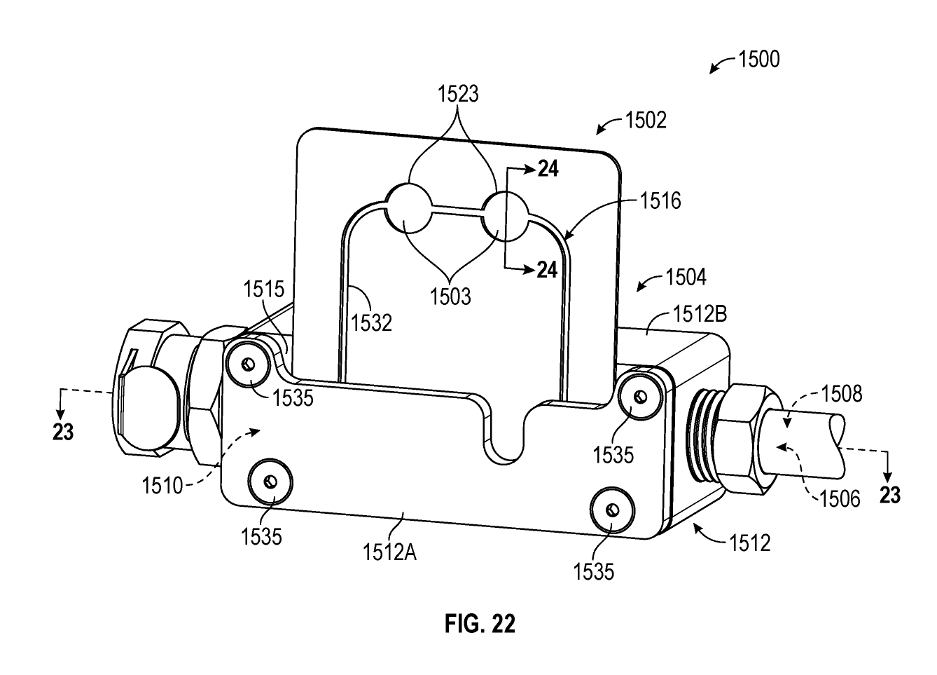

As shown in FIGS. 21-23, the adapter 1504 includes a first fluid pathway 1506 that is open-ended (i.e., when not coupled to a reprocessing system), including an inlet 1508 and an outlet 1510. The adapter 1504 can further include a housing 1512 that can include and define at least a portion of the first fluid pathway 1506. As shown in FIG. 21, the housing 1512 can include a first opening 1511, a second opening 1513, and a first portion 1512A (e.g., a face plate) that can be coupled to a second portion 1512B, e.g., by one or more fasteners 1535 (four screws are shown in FIGS. 21 and 22 by way of example only). By way of further example, the second portion 1512B of the housing 1512 forms the main body of the adapter 1504, housing the first fluid pathway 1506.

As further shown in FIGS. 21-23, the housing 1512 can further include a receptacle 1515 dimensioned to receive at least a portion of the cartridge 1502. By way of example only, the receptacle 1415 is formed between the first portion 1512A and the second portion 1512B of the adapter housing 1512.

By way of example only, in the embodiment of FIGS. 21-23, the receptacle 1515 is in the form of an open slot, and the cartridge 1502 is a flat, thin card that is shaped and sized such that at least a portion of the card can be slid into the receptacle 1515. However, it should be understood that other receptacle and card coupling configurations can be employed, as mentioned above with respect to FIGS. 1 and 2.

As shown in FIG. 21, the cartridge 1502 can include one or more orientation features 1540, such that the cartridge 1502 is keyed with respect to the receptacle 1515 so as to permit positioning the cartridge 1502 in the receptacle 1515 in only one orientation.

The cartridge 1502 can include a second fluid pathway 1516 that is open-ended (i.e., when the cartridge 1502 is not coupled to the adapter 1504), includes an inlet 1518 and an outlet 1520 (see FIG. 21), and is configured to be positioned in fluid communication with the first fluid pathway 1506 of the adapter 1504 when at least a portion of the cartridge 1502 is received in the receptacle 1515 of the adapter 1504.

By way of example only, the process monitoring system 1500 has the same configuration as the process monitoring system 1000 of FIG. 12 and the process monitoring system 1400 of FIGS. 16-18, in which the adapter 1504 includes a shunt channel 1530 (see FIG. 18), and the cartridge 1402 includes a challenge channel 1532. The challenge channel 1532 is configured to mimic the resistance to flow of a medical device of interest, e.g., an endoscope, as described above.

In addition, in some embodiments, as shown in FIGS. 21-23, when at least a portion of the cartridge 1502 is received in the receptacle 1515 of the adapter 1504, the second fluid pathway 1516 of the cartridge 1502 can be fluidly coupled to the first fluid pathway 1506 such that the inlet 1518 and the outlet 1520 of the second fluid pathway 1516 of the cartridge 1502 are located between the inlet 1508 and the outlet 1510 of the first fluid pathway 1506 of the adapter 1504, and particularly, between the first and second openings 1511 and 1513 in the housing 1512.

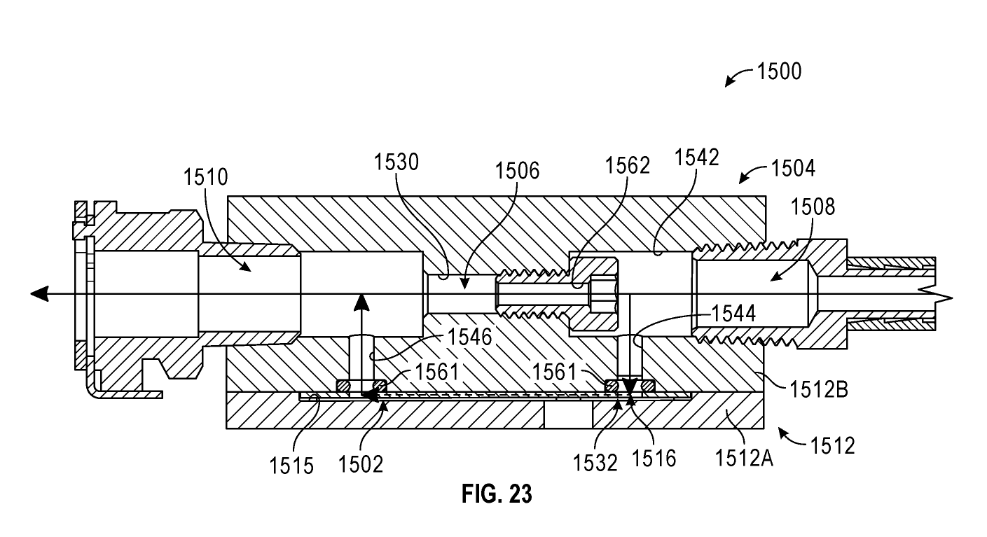

The cartridge 1502 can be movable with respect to the adapter 1504 between (i) a first position (see FIG. 21) in which the cartridge 1502 is not received in the receptacle 1515 of the adapter 1504, and the second fluid pathway 1516 is not in fluid communication with the first fluid pathway 1506 of the adapter 1504; and (ii) a second position (see FIGS. 22 and 23) in which the cartridge 1502 is at least partially received in the receptacle 1515 of the adapter 1504, and the second fluid pathway 1516 is in fluid communication with the first fluid pathway 1506 of the adapter 1504.

A person of ordinary skill in the art will appreciate that the first and second fluid pathways 1506, 1516 can include gaskets, seals and/or valves (e.g., at the inlets 1508, 1518 and outlets 1510, 1520) to control the fluid connection between the first and second fluid pathways 1506, 1516 and to prevent leaks. By way of example, as shown in FIG. 21, the process monitoring system 1500 includes seals 1561 which are positioned to seal around the inlet 1518 and the outlet 1520 of the second fluid pathway 1516.

As shown in FIGS. 21 and 22, the cartridge 1502 includes two indicators 1503 by way of example only, which can include two chemical indicators, two biological indicators, or one of each. As further shown in FIGS. 21 and 22, the indicators 1503 are each located in a chamber 1523 that is in fluid communication with the second fluid pathway 1516, and particularly, with the challenge channel 1532, such that fluid moving through the second fluid pathway 1516 will contact the indicator 1503. In some embodiments, the cartridge 1502 can be transparent or can include a transparent window adjacent the chambers 1523, such that the indicators 1503 are visible or detectable by a reading apparatus, e.g., after being removed from being coupled to the adapter 1404.

As mentioned above, the adapter 1504 includes the shunt channel 1530, and a portion of the fluid flow through the adapter 1504 can be diverted (e.g., sampled) to the cartridge 1502 for monitoring. Because the shunt channel 1530 is configured not to impede fluid flow in a main fluid pathway of a reprocessing system to which the process monitoring system 1500 is coupled, and the challenge channel 1532 is positioned in parallel with the shunt channel 1530, the process monitoring system 1500 does not significantly affect, i.e., increase, the resistance to fluid flow of the reprocessing system. In addition, when a cartridge 1502 is not coupled to the adapter 1504, fluid can flow through the adapter 1504 without being sampled, again, not affecting the overall resistance to flow of the reprocessing system.

Fluid flow through the process monitoring system 1500 will now be described in greater detail with reference to FIG. 23. Fluid flow through the process monitoring system 1500 is generally shown with bold arrows, with a dashed arrow schematically representing the fluid flow through the cartridge 1502, and particularly, the challenge channel 1532 and the chambers 1523 where the indicators 1503 are located. The shunt channel 1530 is sized so as to provide no additional resistance to flow to a reprocessor, such as an AER, e.g., than a native hookup configuration. Located along the first fluid pathway 1506 are diversion ports to and from the cartridge 1502. As a result, in some embodiments, the first fluid pathway 1506 can be described as having (i) a primary path 1542, (ii) a first secondary path 1544 positioned to connect the primary path 1542 to the inlet 1518 (see FIG. 21) of the second fluid pathway 1516 of the cartridge 1502, e.g., when at least a portion of the cartridge 1502 is received in the receptacle 1515 of the adapter 1504, and (iii) a second secondary path 1546 positioned to connect the primary path 1542 to the outlet 1520 (see FIG. 21) of the second fluid pathway 1516, e.g., when at least a portion of the cartridge 1502 is received in the receptacle 1515 of the adapter 1504.

Flow from the primary path 1542 through the second fluid pathway 1516 of the cartridge 1502, e.g., via the first and second secondary paths 1544 and 1546, can be achieved in a variety of ways. By way of example only, flow is generated into and out of the second fluid pathway 1516 using a restriction 1562 (e.g., a restriction orifice) in the primary path 1542. The restriction 1562 can include a point or region in the first fluid pathway 1506 in which the cross-sectional dimension or area is less than that of the input or supply/feeding line, i.e., less than that of the inlet 1508 to the first fluid pathway 1506.

As shown in FIG. 23, fluid can enter the adapter 1504 into a larger chamber located upstream of the restriction 1562, thus causing the fluid to decelerate. Depending on the size of the restriction 1562, a known pressure drop can be created between the first and second secondary paths 1544 and 1546, and particularly, between the ports thereto, causing fluid to flow through the second fluid pathway 1516 of the cartridge 1502. Particularly, in some embodiments, as shown in FIG. 23, the restriction 1562 can be located downstream of first secondary path 1544 to divert fluid flow into the first secondary path 1544.

In some embodiments, the process monitoring system 1500 can further include one or more valves positioned in the first secondary paths 1544 and/or the second secondary path 1546, as described above with respect to FIG. 16.

In use, a user can connect any of the process monitoring systems described above to a reprocessor of interested, e.g., directly to an AER using an appropriate connection harness. After completion of the cycle, the user can remove the cartridge from the adapter (e.g., if necessary to access or read the indicators), e.g., while leaving the adapter coupled to the reprocessor. 5 The user can then visualize or detect a colorimetric response of the chemical indicator (if employed) to establish whether certain desired conditions were met. If a biological indicator was employed, the user can activate the biological indicator by breaking a frangible vial containing growth media allowing media to enter the chamber holding the indicator. The cartridge can then be placed in an incubator, which can also be capable of reading the response from the biological indicator. Depending on the effectiveness of the reprocessing cycle, a response can then be detected at a determined time point to establish whether the cycle passed or failed to meet desired conditions.

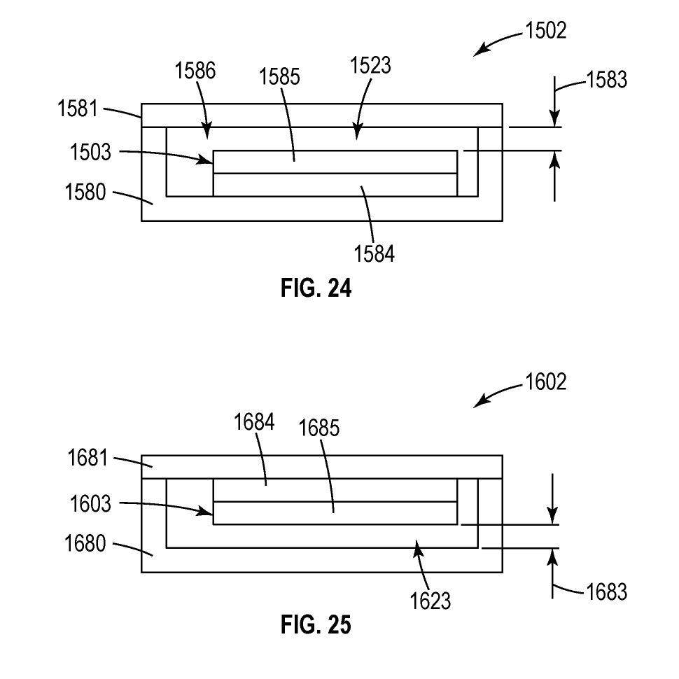

As shown in FIG. 24, the cartridge 1502 can be comprised of one or more layers. For example, the cartridge 1502 can have at least a first layer 1580. The first layer 1580 can have a groove 1586 formed therewith forming a portion of the chamber 1523 and at least a portion of the second fluid pathway. An indicator 1503 can be housed in the chamber 1523. In some embodiments, the indicator 1503 comprises a first substrate with an indicator substance 1585 such as a polyethyleneimine (PEI) indicator disposed therein. For example, the PEI indicator can be adsorbed into a filter paper substrate 1585 and can be further coated with a second substrate 1584 such as a polymer. The indicator 1503 can be allowed to float freely within the chamber 1523 or be coupled to the first layer 1580 by an adhesive.

The cartridge 1502 can also have a second layer 1581 that forms a fluidic seal with the first layer 1580. In some embodiments, the second layer 1581 can be a film (e.g., a cover film) that is transparent. In some embodiments, the indicator substance 1585 of the indicator 1503 can be facing the second layer 1581 to promote viewing. For example, the indicator substance 1585 can react with the liquid disinfectant and can be analyzed through the second layer 1581 by a reading apparatus. The indicator 1503 can be spaced apart from the second layer 1581 at a particular stand-off distance 1583 sufficient to allow liquid disinfectant to contact the indicator substance 1585.

FIG. 25 illustrates a cartridge 1602 having a first layer 1680 and a second layer 1681. A chamber 1623 can be formed from the first layer 1680 and the second layer 1681. The cartridge 1602 can be similar to the cartridge 1502 except that the indicator 1603 is coupled to or disposed on the second layer 1681. The indicator 1603 can have an indicator substance 1685 and a substrate 1684 that is disposed on the second layer 1681. In some embodiments, the substrate 1684 and second layer 1681 is sufficiently transparent to allow a change in the indicator substance 1685 to be viewed from the second layer 1681. The indicator substance 1685 can be oriented toward the second fluid pathway and toward the first layer 1680. The indicator 1603 and the first layer 1680 can have a stand-off distance 1683 to allow liquid disinfectant to flow over the indicator 1603. The configuration of 1602 prevents condensation, which results from rapid cooling, from occurring between the second layer 1681 and the indicator 1603, and thus obviates interference of the reading process due to condensate.

Each embodiment shown in the figures is illustrated as a separate embodiment for clarity in illustrating a variety of features of the cartridges, adapters, and/or process monitoring systems of the present disclosure. However, it should be understood that any combination of elements and features of any of the embodiments illustrated in the figures and described herein can be employed in the cartridges, adapters, and/or process monitoring systems of the present disclosure.

The following embodiments are intended to be illustrative of the present disclosure and not limiting.

### Illustrative Embodiments

1. A cartridge for use with a process monitoring system, the process

monitoring system comprising an adapter comprising a first fluid pathway and configured to be positioned in fluid communication with a reprocessing system, at least a portion of the cartridge configured to be removably received in a receptacle of the adapter, the cartridge comprising:

a second fluid pathway having an inlet and an outlet configured to be positioned in fluid communication with the first fluid pathway of the adapter when at least a portion of the cartridge is positioned in the receptacle of the adapter, such that the second fluid pathway of the cartridge is in fluid communication with the first fluid pathway of the adapter and fluid flow through the first fluid pathway of the adapter is at least partially diverted through the second fluid pathway of the cartridge when at least a portion of the cartridge is received in the receptacle of the adapter, and at least one indicator positioned on the cartridge in fluid communication with the second fluid pathway of the cartridge.

2. A process monitoring system comprising:
an adapter comprising:
   a first fluid pathway and configured to be positioned in fluid communication with a reprocessing system, the first fluid pathway having an inlet and an outlet, and
   a receptacle; and
the cartridge of embodiment 1.

3. The cartridge of embodiment 1 or the process monitoring system of embodiment 2, wherein the indicator is located in a chamber positioned in fluid communication with the second fluid pathway.

4. The cartridge of embodiment 1 or 3 or the process monitoring system of embodiment 2 or 3, wherein at least one of the first fluid pathway and the second fluid pathway includes a shunt channel

5. The cartridge of any of embodiments 1 and 3-4 or the process monitoring system of any of embodiments 2-4, wherein at least one of the first fluid pathway and the second fluid pathway includes a challenge channel

6. The cartridge of any of embodiments 1 and 3-5 or the process monitoring system of any of embodiments 2-5, wherein the first fluid pathway of the cartridge includes a shunt channel

7. The cartridge of any of embodiments 1 and 3-6 or the process monitoring system of any of embodiments 2-6, wherein the first fluid pathway of the cartridge includes a challenge channel

8. The cartridge of any of embodiments 1 and 3-7 or the process monitoring system of any of embodiments 2-7, wherein the second fluid pathway of the adapter includes a shunt channel

9. The cartridge of any of embodiments 1 and 3-8 or the process monitoring system of any of embodiments 2-8, wherein the second fluid pathway of the adapter includes a challenge channel

10. The cartridge of any of embodiments 1 and 3-9 or the process monitoring system of any of embodiments 2-9, wherein the first fluid pathway of the cartridge includes a shunt channel and the second fluid pathway of the adapter includes a challenge channel

11. The cartridge of any of embodiments 1 and 3-10 or the process monitoring system of any of embodiments 2-10, wherein the first fluid pathway of the cartridge includes a challenge channel and the second fluid pathway of the adapter includes a shunt channel

12. The cartridge of any of embodiments 1 and 3-11 or the process monitoring system of any of embodiments 2-11, wherein the first fluid pathway of the cartridge includes a shunt channel and a challenge channel

13. The cartridge of any of embodiments 1 and 3-12 or the process monitoring system of any of embodiments 2-12, wherein the second fluid pathway of the adapter includes a shunt channel and a challenge channel

14. The cartridge of any of embodiments 1 and 3-13 or the process monitoring system of any of embodiments 2-13, wherein the first fluid pathway is open-ended when the adapter is not coupled to a reprocessing system.

15. The cartridge of any of embodiments 1 and 3-14 or the process monitoring system of any of embodiments 2-14, wherein the second fluid pathway is open-ended when the cartridge is not received in the receptacle of the adapter.

16. The cartridge of any of embodiments 1 and 3-15 or the process monitoring system of any of embodiments 2-15, wherein the indicator includes at least one of a chemical indicator and a biological indicator.

17. The cartridge of any of embodiments 1 and 3-16 or the process monitoring system of any of embodiments 2-16, wherein the indicator includes a chemical indicator configured to determine whether a liquid disinfectant of the reprocessing system achieved a suitable concentration and a suitable temperature for a suitable period of time.

18. The cartridge of any of embodiments 1 and 3-17 or the process monitoring system of any of embodiments 2-17, wherein the housing of the adapter further includes a first opening that defines the inlet of the first fluid pathway and a second opening that defines the outlet of the first fluid pathway.

19. The cartridge of any of embodiments 1 and 3-18 or the process monitoring system of any of embodiments 2-18, wherein, when at least a portion of the cartridge is received in the receptacle of the adapter, the second fluid pathway of the cartridge is fluidly coupled to the first fluid pathway such that the inlet and the outlet of the second fluid pathway of the cartridge are located between the inlet and the outlet of the first fluid pathway of the adapter.

20. The cartridge of any of embodiments 1 and 3-19 or the process monitoring system of any of embodiments 2-19, wherein the receptacle of the adapter is a slot, and the cartridge is a card dimensioned to be at least partially received in the slot.

21. The cartridge of any of embodiments 1 and 3-20 or the process monitoring system of any of embodiments 2-20, wherein the cartridge is a card having a thickness of no greater than 25 mm.

22. The cartridge of any of embodiments 1 and 3-21 or the process monitoring system of any of embodiments 2-21, wherein the cartridge is movable between a first position in which the cartridge is not received in the receptacle of the adapter and the second fluid pathway is not in fluid communication with the first fluid pathway and a second position in which at least a portion of the cartridge is received in the receptacle of the adapter and the second fluid pathway is in fluid communication with the first fluid pathway.

23. The cartridge of any of embodiments 1 and 3-22 or the process monitoring system of any of embodiments 2-22, wherein the cartridge is movable between a first position in which the second fluid pathway is not in fluid communication with the first fluid pathway and a second position is which the second fluid pathway is in fluid communication with the first fluid pathway.

24. The cartridge of any of embodiments 1 and 3-23 or the process monitoring system of any of embodiments 2-23, wherein the first fluid pathway includes a gap, such that the first fluid pathway is incomplete when the cartridge is not received in the receptacle of the adapter, and wherein the gap is filled by at least a portion of the second fluid pathway when at least a portion of the cartridge is received in the receptacle of the adapter.

25. The cartridge of any of embodiments 1 and 3-24 or the process monitoring system of any of embodiments 2-24, wherein the first fluid pathway of the adapter includes a restriction.

26. The cartridge of any of embodiments 1 and 3-25 or the process monitoring system of any of embodiments 2-25, wherein the first fluid pathway of the adapter includes a venturi.

27. The cartridge of any of embodiments 1 and 3-26 or the process monitoring system of any of embodiments 2-26, wherein the first fluid pathway of the adapter includes a primary path, a first secondary path positioned to connect the primary path to the inlet of the second fluid pathway, and a second secondary path positioned to connect the primary path to the outlet of the second fluid pathway.

28. The cartridge or the process monitoring system of embodiment 27, further comprising a first valve positioned in the first secondary path and a second valve positioned in the second secondary path.

29. The cartridge or the process monitoring system of embodiment 27 or 28, wherein the primary path includes a restriction located downstream of the first secondary path.

30. The cartridge or the process monitoring system of any of embodiments 27-29, wherein the primary path includes a venturi located upstream of the first secondary path.

31. The cartridge or the process monitoring system of embodiment 30, wherein the primary path includes a venturi located downstream of the second secondary path.

32. An endoscope reprocessing system comprising:
the process monitoring system of any of embodiments 2-31; and
a fluid pathway configured to be positioned in fluid communication with the process monitoring system and an endoscope.

33. The endoscope reprocessing system of embodiment 32, wherein the process monitoring system and the endoscope are fluidly coupled in series.

34. The endoscope reprocessing system of embodiment 32 or 33, wherein the process monitoring system and the endoscope are fluidly coupled in parallel.

35. The endoscope reprocessing system of any of embodiments 32-34, wherein the endoscope reprocessing system has a resistance to fluid flow that is increased by the process monitoring system by no greater than 20%.

36. The endoscope reprocessing system of any of embodiments 32-35, further comprising a basin for receiving an endoscope.

37. The endoscope reprocessing system of embodiment 36, wherein the adapter is located outside of the basin.

38. A process monitoring system comprising:
an adapter comprising:
a first fluid pathway and configured to be positioned in fluid communication with a reprocessing system, the first fluid pathway having an inlet and an outlet, and
a housing comprising a receptacle; and

a cartridge, at least a portion of the cartridge configured to be removably received in the receptacle of the adapter, the cartridge comprising:
a second fluid pathway having an inlet and an outlet configured to be positioned in fluid communication with the first fluid pathway of the adapter when at least a portion of the cartridge is positioned in the receptacle of the adapter, such that the second fluid pathway of the cartridge is in fluid communication with the first fluid pathway of the adapter and fluid flow through the first fluid pathway of the adapter is at least partially diverted through the second fluid pathway of the cartridge when at least a portion of the cartridge is received in the receptacle of the adapter, and
at least one indicator positioned on the cartridge in fluid communication with the second fluid pathway of the cartridge.

39. The cartridge of any of embodiments 1 and 3-26 or the process monitoring system of any of embodiments 2-26, wherein the chemical indicator is disposed on a substrate.

40. The cartridge of any of embodiments 1 and 3-26, and 39 or the process monitoring system of any of embodiments 2-26 and 39, wherein the chemical indicator is polyethyleneimine.

41. The cartridge of any of embodiments 1 and 3-26, and 39-40 or the process monitoring system of any of embodiments 2-26 and 39-40, wherein a chamber in fluid communication with the second fluid pathway is formed from at least a first layer of the cartridge.

42. The cartridge of any of embodiments 1 and 3-26, and 39-41 or the process monitoring system of any of embodiments 2-26 and 39-41, wherein the chamber in fluid communication with the second fluid pathway is formed from at least a second layer of the cartridge.

43. The cartridge of any of embodiments 1 and 3-26, and 39-42 or the process monitoring system of any of embodiments 2-26 and 39-42, wherein the second layer is a film.

44. The cartridge of any of embodiments 1 and 3-26, and 39-43 or the process monitoring system of any of embodiments 2-26 and 39-44, wherein the second layer is transparent and is adjacent the chamber, such that the chemical or biological indicator is visible or detectable by a reading apparatus.

45. The cartridge of any of embodiments 1 and 3-26, and 39-44 or the process monitoring system of any of embodiments 2-26 and 39-44, wherein the chemical indicator is disposed on the second layer.

46. The cartridge of embodiment 45, wherein the chemical indicator is oriented facing the second fluid pathway.

47. The cartridge of embodiment 45, wherein the chemical indicator is oriented facing the second layer.

48. The cartridge of embodiment 45, wherein the chemical indicator is oriented facing the first layer.

It is to be understood that the invention is not limited in its application to the details of construction and the arrangement of components set forth in the above description or illustrated in the accompanying drawings. The invention is capable of other embodiments and of being practiced or of being carried out in various ways. Also, it is to be understood that the phraseology and terminology used herein is for the purpose of description and should not be regarded as limiting. It is to be further understood that other embodiments may be utilized, and structural or logical changes may be made without departing from the scope of the present disclosure.

The following working and prophetic examples are intended to be illustrative of the present disclosure and not limiting.

### EXAMPLES

### Prophetic Examples 1-18

Prophetic Examples 1-18 of the present disclosure are described in Table 1, which details various features and configurations of process monitoring systems of the present disclosure, with reference to FIGS. 5-18 and 21-23. Note that FIGS. 7-15 do not illustrate the first column of Table 1—i.e., whether the system fluid connection mode to a main fluid pathway of a reprocessing system is serial or parallel. As a result, FIGS. 7-15 can apply to both the Serial and Parallel fluid connection modes of the first column.

TABLE 1
Features and Configurations of Prophetic Examples 1-18

| | System Fluid | | | | | |
|---|---|---|---|---|---|---|
| | Connection | | | | | |
| | Mode in | | | | | |
| | Relationship | | | | Location | Repre- |
| | to Main Fluid | Challenge | Challenge | Shunt | of | sen- |
| Ex. | Pathway of | Channel | Channel | Channel | Shunt | tative |
| No. | Reprocessor | Present | Location | Present | Channel | Figures |
| 1 | Serial | No | NA | No | NA | 5, 7 |
| 2 | Serial | No | NA | Yes | Cartridge | 5, 8 |
| 3 | Serial | No | NA | Yes | Adapter | 5, 9 |
| 4 | Parallel | No | NA | No | NA | 6, 7 |
| 5 | Parallel | No | NA | Yes | Cartridge | 6, 8 |
| 6 | Parallel | No | NA | Yes | Adapter | 6, 9 |
| 7 | Serial | Yes | Cartridge | No | NA | 5, 10 |
| 8 | Serial | Yes | Adapter | No | NA | 5, 11 |
| 9 | Serial | Yes | Cartridge | Yes | Adapter | 5, 12, |
| | | | | | | 16-18, |
| | | | | | | 21-23 |
| 10 | Serial | Yes | Adapter | Yes | Cartridge | 5, 13 |
| 11 | Serial | Yes | Adapter | Yes | Adapter | 5, 14 |
| 12 | Serial | Yes | Cartridge | Yes | Cartridge | 5, 15 |
| 13 | Parallel | Yes | Cartridge | No | NA | 6, 10 |
| 14 | Parallel | Yes | Adapter | No | NA | 6, 11 |
| 15 | Parallel | Yes | Cartridge | Yes | Adapter | 6, 12, |
| | | | | | | 16-18, |
| | | | | | | 21-23 |
| 16 | Parallel | Yes | Adapter | Yes | Cartridge | 6, 13 |
| 17 | Parallel | Yes | Adapter | Yes | Adapter | 6, 14 |
| 18 | Parallel | Yes | Cartridge | Yes | Cartridge | 6, 15 |

### Working Example 19

Example 19 was fabricated according to the descriptions given above, shown in detail in FIGS. 16-18, and further described in Table 1 as Examples 9 and 15. The cartridge was prepared by injection molding a card-shaped structure with the following design features: inlet and outlet ports that interface with the reusable adapter; a challenge channel; an indicator chamber to house a chemical indicator (CI); a baffling section transitioning the fluid flow from the challenge channel to the indicator chamber; and two semicircular indentations on the outlet side of the indicator chamber to promote turbulent flow within the chamber. The cartridge material was medical grade acrylonitrile butadiene styrene (ABS). The top surface of the cartridge was injection molded to provide open grooved structures (inlet, outlet, challenge channel, indicator chamber) that were covered by laminating a single sheet of 0.254 mm thick polyester film backing with a silicone pressure sensitive adhesive over the entire top side of the cartridge. The cartridge was 1.524 mm thick. The challenge channel had a rectangular cross section with dimensions of 1.524 mm wide by 1.016 mm deep. The challenge channel had a length of approximately 156 mm (including the baffling section).

The adapter was machined from anodized aluminum and was designed to receive the disposable cartridge into a receptacle slot on the adapter. The adapter was also designed to connect directly to a MEDIVATORS® DSD-201LT dual basin AER outlet port via the male CPC connector. This male connector was attached to the body of the adapter through a short section of flexible plastic tubing. The tubing size (3.175 mm or ⅛ inch ID) was exactly the same as that used in the various MEDIVATORS® hookup harnesses used to connect an endoscope to the AER. This short section of flexible tubing allowed the adapter (and cartridge, when inserted) to be placed in the AER basin. At the opposite end of the adapter body was a female CPC connector matching the exact size of the outlet connector used in the AER basin. This allowed connection of the adapter to a given hookup harness without having to make any modifications to the harness. The adapter was designed to have a main channel or shunt in which liquid can flow directly from the output port in the basin of the AER to the connection harness and ultimately the endoscope being reprocessed. This shunt channel radius was approximately 3.17 mm in diameter and was sized to provide no additional resistance to flow. Two parallel side passages (i.e., first and second secondary paths) were designed into the adapter and were perpendicularly connected to the shunt channel The first secondary path allowed flow of disinfectant from the shunt channel (or primary path) to the inlet port of the cartridge. The second secondary path received flow from the cartridge outlet port and thus returned the liquid flow to the primary path of the adapter.

The adapter further included a plunger mechanism which opened and closed the inlet and outlet ports of the secondary paths of the adapter. When the cartridge was not present, the plungers pushed a pair of cone seals against the face plate of the adapter to prevent liquid from leaking. When the cartridge was inserted into the slot of the adapter, the plunger's springs were compressed and allowed the cartridge to slide into the adapter receptacle between the body and the faceplate of the adapter. When the cartridge was fully seated into the receptacle, the secondary paths were positioned directly over the inlet and outlet ports of the cartridge, providing a fluidic connection. In this state, a portion of the liquid stream flowing through the adapter entered the cartridge and flowed through the challenge channel and the indicator chamber, exited the cartridge, and ultimately reentered the primary path of the adapter.

The embodiments described above and illustrated in the figures are presented by way of example only and are not intended as a limitation upon the concepts and principles of the present disclosure. As such, it will be appreciated by one having ordinary skill in the art that various changes in the elements and their configuration and arrangement are possible without departing from the spirit and scope of the present disclosure.

All references and publications cited herein are expressly incorporated herein by reference in their entirety into this disclosure.

Various features and aspects of the present disclosure are set forth in the following claims.

### Working Example 20

A paper substrate (available under the trade designation Whatman from Whatman PLC, United Kingdom) coated with a film laminate (available under the trade designation Surlyn from Dupont, Wilimington, Del.) was coated (on the paper side) with an indicator composition comprising a branched poly(ethyleneimine) (60,000 MW, available from Sigma Aldrich, St. Louis, Mo.) at 10% solids in water. The indicator-coated substrate was dried at 100° C. for 5 minutes. The indicator-coated substrate was positioned in the bottom of the indicator chamber of the cartridge of working example 19 such that the indicator-coated surface faced away from the chamber floor and toward disinfectant fluid according to FIG. 24. Upon exposure to 0.35% ortho-phthalaldehyde fluid in an automatic endoscope reprocessor (AER) (commercially available as Medivators DSD-201 from Medivators, Minneapolis, Minn.) using ortho-phthalaldehyde as high-level disinfectant, the indicator color changed from white to yellow-orange. After subsequently placing the cartridge into a refrigerator for 5 minutes, condensation could be seen over the indicator, which obscured visualization and assessment of the indicator color.

### Working Example 21

A cartridge was prepared as in Example 20, except that the indicator-coated substrate was adhered to the polyester cover film for the cartridge such that the film laminate contacted the adhesive layer of the cover film and the coated paper faced toward the chamber floor according to FIG. 25. Upon exposure to 0.35% ortho-phthalaldehyde disinfectant in an AER, the indicator color changed from white to yellow-orange. After subsequently placing the cartridge into a refrigerator for 5 minutes, no condensation could be seen to be obscuring the visual assessment of the indicator.

1. A cartridge for use with a process monitoring system, the process monitoring system comprising an adapter comprising a first fluid pathway and configured to be positioned in fluid communication with a reprocessing system, at least a portion of the cartridge configured to be removably received in a receptacle of the adapter, the cartridge comprising:
a second fluid pathway having an inlet and an outlet configured to be positioned in fluid communication with the first fluid pathway of the adapter when at least a portion of the cartridge is positioned in the receptacle of the adapter, such that the second fluid pathway of the cartridge is in fluid communication with the first fluid pathway of the adapter and fluid flow through the first fluid pathway of the adapter is at least partially diverted through the second fluid pathway of the cartridge when at least a portion of the cartridge is received in the receptacle of the adapter, and
at least one indicator positioned on the cartridge in fluid communication with the second fluid pathway of the cartridge.

2. (canceled)

3. The cartridge of claim 1, wherein at least one of the first fluid pathway and the second fluid pathway includes a shunt channel.

4. The cartridge of claim 1, wherein at least one of the first fluid pathway and the second fluid pathway includes a challenge channel.

5. The cartridge of claim 1, wherein the first fluid pathway of the cartridge includes a shunt channel and the second fluid pathway of the adapter includes a challenge channel.

6. The cartridge of claim 1, wherein the first fluid pathway of the cartridge includes a challenge channel and the second fluid pathway of the adapter includes a shunt channel.

7. The cartridge of claim 1, wherein the indicator includes at least one of a chemical indicator and a biological indicator.

8. (canceled)

9. The cartridge of claim 1, wherein the cartridge is a card having a thickness of no greater than 25 mm.

10. The cartridge of claim 1, wherein the cartridge is movable between a first position in which the second fluid pathway is not in fluid communication with the first fluid pathway and a second position is which the second fluid pathway is in fluid communication with the first fluid pathway.

11. The cartridge of claim 1, wherein the first fluid pathway includes a gap, such that the first fluid pathway is incomplete when the cartridge is not received in the receptacle of the adapter, and wherein the gap is filled by at least a portion of the second fluid pathway when at least a portion of the cartridge is received in the receptacle of the adapter.

12. The cartridge of claim 1, wherein the first fluid pathway of the adapter includes a restriction.

13.-14. (canceled)

15. The cartridge of claim 1, wherein the cartridge comprises a first layer and a second layer forming a chamber therein, the chamber is in fluid communication with the second fluid pathway, wherein the second layer is transparent and is adjacent the chamber.

16. The cartridge of claim 7, wherein the chemical indicator is disposed on the second layer oriented facing the second fluid pathway.

17.-20. (canceled)

21. A process monitoring system comprising:
an adapter comprising:
a first fluid pathway and configured to be positioned in fluid communication with a reprocessing system, the first fluid pathway having an inlet and an outlet, and
a receptacle; and
a cartridge comprising:
a second fluid pathway having an inlet and an outlet configured to be positioned in fluid communication with the first fluid pathway of the adapter when at least a portion of the cartridge is positioned in the receptacle of the adapter, such that the second fluid pathway of the cartridge is in fluid communication with the first fluid pathway of the adapter and fluid flow through the first fluid pathway of the adapter is at least partially diverted through the second fluid pathway of the cartridge when at least a portion of the cartridge is received in the receptacle of the adapter, and
at least one indicator positioned on the cartridge in fluid communication with the second fluid pathway of the cartridge.

22. The process monitoring system of claim 21, wherein the first fluid pathway of the adapter includes a venturi.

23. The process monitoring system of claim 21, wherein the first fluid pathway of the adapter includes a primary path, a first secondary path positioned to connect the primary path to the inlet of the second fluid pathway, and a second secondary path positioned to connect the primary path to the outlet of the second fluid pathway.

24. An endoscope reprocessing system comprising:
a process monitoring system comprising:
an adapter comprising:
a first fluid pathway and configured to be positioned in fluid communication with a reprocessing system, the first fluid pathway having an inlet and an outlet, and
a receptacle; and
a cartridge comprising:
a second fluid pathway having an inlet and an outlet configured to be positioned in fluid communication with the first fluid pathway of the adapter when at least a portion of the cartridge is positioned in the receptacle of the adapter, such that the second fluid pathway of the cartridge is in fluid communication with the first fluid pathway of the adapter and fluid flow through the first fluid pathway of the adapter is at least partially diverted through the second fluid pathway of the cartridge when at least a portion of the cartridge is received in the receptacle of the adapter, and
at least one indicator positioned on the cartridge in fluid communication with the second fluid pathway of the cartridge; and
a fluid pathway configured to be positioned in fluid communication with the process monitoring system and an endoscope.

25. The endoscope reprocessing system of claim 24, wherein the process monitoring system and the endoscope are fluidly coupled in series.

26. The endoscope reprocessing system of claim 24, wherein the process monitoring system and the endoscope are fluidly coupled in parallel.

27. The endoscope reprocessing system of claim 24, wherein the endoscope reprocessing system has a resistance to fluid flow that is increased by the process monitoring system by no greater than 20%.
